(19) United States
(12) Patent Application Publication
(10) Pub. No.: US 2024/0241023 A1
(43) Pub. Date: Jul. 18, 2024

(54) AUTOMATED METHODS AND SYSTEMS FOR RAPID IDENTIFICATION OF TARGET PARTICLES

(71) Applicant: CAP DIAGNOSTICS, LLC DBA PATHNOSTICS, Irvine, CA (US)

(72) Inventors: David A. Baunoch, Irvine, CA (US); Tim Bruemmer, Irvine, CA (US); Michael L. Opel, Irvine, CA (US); Natalie Luke, Irvine, CA (US); David Flaten, Irvine, CA (US); Richard Festa, Irvine, CA (US); Marcy Akhlaghpour, Irvine, CA (US); Scott Atkin, Irvine, CA (US)

(21) Appl. No.: 18/620,436

(22) Filed: Mar. 28, 2024

Related U.S. Application Data
(63) Continuation-in-part of application No. PCT/US22/77477, filed on Oct. 3, 2022.
(60) Provisional application No. 63/251,433, filed on Oct. 1, 2021.
(60) Provisional application No. 63/329,757, filed on Apr. 11, 2022.
(60) Provisional application No. 63/493,416, filed on Mar. 31, 2023.

(51) Int. Cl. G01N 15/0205; C12Q 1/18; G01N 35/00

(52) U.S. Cl. CPC G01N 15/0205; C12Q 1/18; G01N 35/00584

(57) ABSTRACT

Flow cytometry-based methods and systems for rapidly identifying a target particle, such as a target cell, a target particle, or multiple distinct target cells or target particles. The methods and systems herein are capable of distinguishing between and identifying multiple distinct target cells or target particles from a single sample containing a plurality of targets. The methods and systems herein can be used for applications such as but not limited to identification and quantification of infectious agents, e.g., bacteria, viruses, fungi, parasites, etc., in a sample.

### CROSS-REFERENCES TO RELATED APPLICATIONS

This application is a continuation-in-part and claims benefit of International Application No. PCT/US22/77477, filed Oct. 3, 2022, which claims benefit of U.S. Provisional Application No. 63/329,757 filed Apr. 11, 2022, and U.S. Provisional Application No. 63/251,433 filed Oct. 1, 2021, the specifications of which are incorporated herein in their entirety by reference.

This application is also a nonprovisional application and claims benefit of U.S. Provisional Application No. 63/493,416 filed Mar. 31, 2023, the specification of which is incorporated herein in their entirety by reference.

### FIELD OF THE INVENTION

The present invention is related to automated flow cytometry-based methods and systems for rapid detection of target particles, e.g., cells or other particles. The methods and systems herein may be applied to rapid identification of infectious agents (e.g., cells, viral particles, etc.) in samples. The systems may also integrate automated antibiotic susceptibility testing and other aspects of sample preparation, processing, and/or incubation.

### BACKGROUND OF THE INVENTION

Infectious diseases can affect multiple organs systems and are responsible for significant morbidity, mortality, and economic impact.

The present invention features flow cytometry-based methods and systems for rapidly identifying target particles such as a cell or other particles, or multiple distinct target particles (e.g., cells or other particles) in a sample containing a plurality of targets. For example, the present invention features flow cytometry-based methods and systems for rapidly identifying one or more infectious agents (e.g., cells, viral particles, etc.) in a sample. The systems and methods may also feature quantifying said target particles (e.g. cells or other particles such as but not limited to infectious agents). The system may also feature, identifying the presence of an active infection and/or determining if the infectious agents are alive. Detection of whether or not the infectious agents are alive may be achieved using a separate label, distinguishable from the other labels used in the assay, for identification. In certain embodiments, the system has the ability to perform and integrate antibiotic susceptibility testing and/or other aspects of sample preparation, processing, and/or incubation.

The present invention features methods and flow cytometry systems wherein the data analysis utilizes fluorophore-labeled antibodies specific to antigens which appear in abundance preferentially on each one of the various target particles of interest (e.g., cells or other particles, such as but not limited to infectious agents of interest). While the present invention discloses the term “fluorophore” in detail, the use of the term “fluorophore” in present invention includes detectable moieties such small organic dyes, tandem dyes, polymer dyes, phycobiliproteins, etc., and the present invention is not limited to said detectable moieties. The use of quantum dots, other dyes, or other appropriate detectable moieties is within the scope of the present invention.

As will be described herein, the identification of the target particle (e.g., cell, particle, etc.) is based on the identification of the dominantly abundant antibody conjugate. For example, using one particular approach further described below in detail, the dominant antibody-fluorophore conjugate (in an event) has an abundance that is greater than X times any of the other abundances representing antibody conjugates for other target particles, wherein X represents a minimum abundance ratio and is associated with the antibody. X may be, for example, at least >1, or approximately 2, 3, 4, 5, 6, 7, 8, 9, 10, 10+, etc.

### BRIEF SUMMARY OF THE INVENTION

The present invention features flow cytometry-based methods and systems for rapidly identifying one or more target particles, such as a cell or other particle, in a sample. For example, the present invention provides flow cytometry systems and methods for identifying one or more infectious agents in a sample.

For example, the present invention features a system configured to analyze optical data captured by a flow cytometer, wherein the data captured by the flow cytometer is from a detection system therein that detects optical emission as a target particle passes through the flow cytometer. The optical emission is derived from one or a combination of: (i) one or more fluorophores each conjugated to an antibody bound to the target particle, the antibody being one of n antibody species each specific for one of n target particles, the one or more fluorophores being from a group of m total fluorophores used for labeling the n antibody species forming a pool of l antibody-fluorophore conjugates, and (ii) potential autofluorescence associated with the target particle. In some embodiments, the system comprises a processor; and a memory component communicatively connected (e.g., operatively connected) to the processor and configured to store an application (e.g., computer readable instructions), wherein the application allows the processor to identify a dominant antibody-fluorophore conjugate on each target particle using an unmixing algorithm, the dominant antibody-fluorophore conjugate being an antibody-fluorophore conjugate that appears in abundance preferentially on the target particle due to its binding to the target particle. In some embodiments, the unmixing algorithm either: uses a single minimum abundance ratio (X) value using the smallest minimum value of X for any of the antibody-fluorophore conjugates constraining the solution to require a dominant antibody fluorophore-conjugate with abundance value of at least X times that of the next most abundant antibody-fluorophore conjugate as a requirement for an acceptable solution for a closed form unmixing algorithm or as a constraint on a numerical mixing algorithm; or partitions the analysis into n separate instances, wherein n is the number of possible target particles that can be identified in the analysis, wherein each instance has a unique minimum abundance ratio requirement based on a predetermined value of X for each antibody-fluorophore conjugate, the minimum abundance ratio requirement being a requirement that a particular dominant antibody-fluorophore conjugate have an abundance value greater than X times that of the next most abundant antibody-fluorophore conjugate in order to be a correct solution for a closed form expression and wherein the minimum abundance ratio requirement is an additional constraint for a numerical solution with target particle identification based on the instance with the smallest residual error. In some embodiments, the memory component is communicatively connected to the processor via a wire connection. In other embodiments, the memory component is communicatively connected to the processor via a wireless connection.

The abundance value for the dominant antibody-fluorophore conjugate is generally required to be above a predetermined threshold level of fluorescence. The residual error of the numerical analysis is generally below a threshold value or below a specific fraction of a next closest residual error value in order to be considered a valid unambiguous result.

The systems described herein are capable of detecting and quantifying a plurality of different targets, e.g., at least 10 different targets, at least 15 different targets, at least 20 different targets, at least 25 different targets, etc., within a single sample. In some embodiments, the minimum abundance ratio (X) is at least >1 for each antibody-fluorophore conjugate. In some embodiments, the minimum abundance ratio (X) is from 1 to 10 for at least one antibody-fluorophore conjugate. In some embodiments, the minimum abundance ratio (X) is from 3 to 10 for at least one antibody-fluorophore conjugate. In some embodiments, the minimum abundance ratio (X) is from 5 to 20 for at least one antibody-fluorophore conjugate.

In some embodiments, the solutions are constrained so that all abundance values are greater than zero. In some embodiments, each target particle has a predetermined autofluorescence signature. In some embodiments, this autofluorescence signature is added as an additional fluorophore paired with the respective minimum abundance ratio (X) for the target particle in that instance. The autofluorescence signatures may be added as an additional fluorophore for purposes of fitting measured spectra in the numerical analysis.

In some embodiments, each antibody is conjugated with a single unique fluorophore. In some embodiments, the pool of l antibody-fluorophore conjugates comprises n different antibodies each labeled with a unique fluorophore. In some embodiments, the pool of l antibody-fluorophore conjugates comprises n different antibodies, a portion of which are each labeled with one fluorophore and a portion of which are each labeled with two or more fluorophores. In some embodiments, in the pool of l antibody-fluorophore conjugates at least one antibody has at least r different antibody conjugates each of which are labeled with one each of r different fluorophores, and said fluorophores form a set that are also used as labels in different combinations on other antibodies for different target particles. In some embodiments, the system can identify a maximum number of particles equal to the sum of all of the terms m!/((m−k)!*k!) for all k from 1 to m using m fluorophores. In some embodiments, the system can identify 3 distinct target particles using 2 fluorophores. In some embodiments, the system can identify 7 distinct target particles using 3 fluorophores. In some embodiments, the minimum abundance ratio requirement is that each of the k fluorophores used to label an antibody for a particular target has an abundance ratio to all other m−k fluorophores that is greater than approximately X/k and wherein X is the minimum abundance ratio for the target particle when all of its target antibody is labeled with a single fluorophore.

In some embodiments, the flow cytometer is a spectral flow cytometry system. In some embodiments, the system is communicatively connected (e.g., operatively connected) to the flow cytometer. In some embodiments, the system is integrated with the flow cytometer. In some embodiments, the target particle is a cell or particle. In some embodiments, the cell or particle is or is associated with an infectious agent. In some embodiments, the infectious agent is a bacterium, a virus, a fungus, or a parasite. In some embodiments, the antibodies are each specific for an infectious agent. In some embodiments, the antibodies are monoclonal antibodies. In some embodiments, the system is communicatively connected to the flow cytometer via a wire connection. In other embodiments, the system is communicatively connected to the flow cytometer via a wireless connection.

Similarly, the present invention features a system configured to analyze optical data captured by a flow cytometer, wherein the data captured by the flow cytometer is from a detection system therein that detects optical emission as a particle passes through the flow cytometer, the optical emission being derived from one or a combination of: (i) one or more fluorophores each conjugated to an antibody bound to the particle, the one or more fluorophores being from a pool of l antibody-fluorophore conjugates, and (ii) autofluorescence associated with the particle. The system comprises a processor; and a memory component communicatively connected (e.g., operatively connected) to the processor and configured to store an application (e.g., computer readable instructions). The application configures the processor to identify the dominant fluorophores on each target particle using an unmixing algorithm, the dominant k fluorophores being the fluorophores that appears in abundance preferentially on the target particle, wherein k is the number of fluorophore-antibody conjugates used to label a particular target and wherein k can be a value from 1 to r wherein r is the number of a set of fluorophores used in various unique combinations to label any single target and r is less than or equal to m. The unmixing algorithm partitions the analysis into n separate instances, wherein n is the number of possible target particles that can be identified in the analysis, wherein each instance has a unique minimum abundance ratio requirement based on a predetermined value of X for each antibody-fluorophore conjugate, the minimum abundance ratio requirement being a requirement that a particular dominant fluorophore conjugate has an abundance value greater than X times that of the next most abundant fluorophore in order to be a correct solution for a closed form expression and wherein the minimum abundance ratio requirement is an additional constraint for a numerical solution. In some embodiments, the memory component is communicatively connected to the processor via a wire connection. In other embodiments, the memory component is communicatively connected to the processor via a wireless connection.

Similarly, the present invention features a system configured to analyze optical data captured by a flow cytometer, wherein the data captured by the flow cytometer is from a detection system therein that detects optical emission as a particle passes through the flow cytometer, the optical emission being derived from one or a combination of: (i) one or more fluorophores each conjugated to an antibody bound to the particle, the one or more fluorophores being from a pool of l antibody-fluorophore conjugates, and (ii) autofluorescence associated with the particle. The system comprises a processor; and a memory component communicatively connected (e.g., operatively connected) to the processor and configured to store an application (e.g., computer readable instructions). The application configures the processor to identify a dominant fluorophore on each target particle using an unmixing algorithm, the dominant fluorophore being a fluorophore that appears in abundance preferentially on the particle. The unmixing algorithm uses a single minimum abundance ratio (X) value using the smallest minimum value of X for any of the antibody-fluorophore conjugates constraining the solution to require a dominant antibody fluorophore-conjugate with abundance value of at least X times that of the next most abundant antibody-fluorophore conjugate as a requirement for an acceptable solution for a closed form unmixing algorithm or as a constraint on a numerical mixing algorithm. In some embodiments, the memory component is communicatively connected to the processor via a wire connection. In other embodiments, the memory component is communicatively connected to the processor via a wireless connection.

The present invention also features antibody-fluorophore conjugates for use in identifying one or more target particles or target cells in a sample with a system according to the disclosures herein. For example, in some embodiments, the antibody of the antibody-fluorophore conjugate is specific for a particular target particle or target cell and the fluorophore of the antibody-fluorophore conjugate is a fluorophore with an emission spectra further distinguished from other similar fluorophore spectra in the analysis by the autofluorescence of the target particle or target cell. In some embodiments, the antibody of the antibody-fluorophore conjugate is specific for a particular target particle or target cell and the fluorophore of the antibody-fluorophore conjugate is selected so that it is: (a) a weak or dim fluorophore if the antibody binds to its target particle or target cell at a relative high binding density; (b) a strong or bright fluorophore if the antibody binds to its target particle or target cell at a relative low binding density; (c) a medium intensity fluorophore if the typical antibody binding to the target particle or target cell is not at a relative high binding density and not at a relative low binding density. In some embodiments, the antibody of the antibody-fluorophore conjugate is specific for a particular infectious agent and the fluorophore of the antibody-fluorophore conjugate is selected so that it is: (a) a weak or dim fluorophore if the antibody binds to its infectious agent at a relative high binding density; (b) a strong or bright fluorophore if the antibody binds to its infectious agent at a relative low binding density; (c) a medium intensity fluorophore if the typical antibody binding to the infectious agent is not at a relative high binding density and not at a relative low binding density.

The present invention also includes flow cytometry panels for use with the systems described in the present invention, wherein the panel comprises a plurality of antibody-fluorophore conjugates according to the disclosures herein.

The present invention also features an automated system for identifying one or more infectious agents in a sample, wherein the system comprises a flow cytometry unit and a processing system for analyzing data from the flow cytometry unit according to the disclosures herein. In some embodiments, the system further comprises an antibiotic susceptibility testing (AST) unit for determining susceptibility and resistance of the one or more infectious agents in the sample to one or more concentrations of one or more antimicrobial agents. In some embodiments, the AST unit comprises a multiwell plate wherein a plurality of wells are loaded with a particular concentration of one or more antimicrobial agents. In some embodiments, the AST unit further comprises a dispensing system for dispensing a portion of the sample into at least one well of the multiwell plate. In some embodiments, the AST unit is operatively connected to the processing system, the processing system further comprises an application (e.g., computer readable instructions) stored by the memory for execution by the processor, the application determines the order in which wells are analyzed for susceptibility or resistance. In some embodiments, the application determines an order in which wells are analyzed that will likely result in the fewest wells needed to be analyzed in order to get all necessary information for providing a minimum inhibitory concentration (MIC) for one or more of the antimicrobial agents. In some embodiments, the application accesses a database comprising data that provides the application a probability of resistance (p_{R}) for one or more concentrations of the one or more antimicrobial agents. In some embodiments, the system determines which wells to analyze first based on the provided p_{R} values. In some embodiments, the application uses conditional probabilities to minimize the number of wells that need to be analyzed, wherein the application calculates the probability of resistance of the one or more infectious agents to a certain antimicrobial agent (A) at a certain concentration based on the resistance of the one or more infectious agents to another antimicrobial agent (B) at a particular concentration.

In some embodiments, the system further comprises an incubation unit comprising one or more incubators for housing and incubating a portion of the sample. In some embodiments, the system further comprises a preparation unit for accepting the sample and preparing the sample for processing through the flow cytometry unit.

In some embodiments, the system is for identifying an active infection with one or more infectious agents in the sample. In some embodiments, the infectious agent is a bacteria, a virus, a fungus, or a parasite. In some embodiments, the infectious agent is Acinetobacter baumannii, Actinotignum schaalii, Aerococcus urinae, Aerococcus urinae, Alloscardovia omnicolens, Candida albicans, Candida glabrata, Candida parapsilosis, Candida tropicalis, Chlamydia, Citrobacter freundii, Citrobacter koseri, Clostridium difficile, Corynebacterium riegelii, Klebsiella aerogenes, Enterococcus faecalis, Escherichia coli, Klebsiella oxytoca, Klebsiella pneumoniae, Morganella morganii, Mycobacterium tuberculosis, Mycoplasma genitalium, Mycoplasma hominis, Neisseria gonorrhoeae, Pantoea agglomerans, Proteus mirabilis, Providencia stuartii, Pseudomonas aeruginosa, Serratia marcescens, Staphylococcus aureus, coagulase-negative Staphylococcus, Streptococcus agalactiae, Streptococcus pyogenes, Viridans Group Streptococcus, Trichomonas vaginalis, Ureaplasma urealyticum, HHV-6, HHV-7, BK Virus, JC Virus, HSV 1&2, Adenovirus, or CMV.

In some embodiments, the system is configured for parallel processing of samples. The system may be fully automated. In some embodiments, the system can process at least 5, at least 10, at least 20, at least 40, at least 50, etc., samples per day.

In some embodiments, the system processes the sample by dispensing portions into one or more multiwell plates. In some embodiments, the system can transport the multiwell plate between the sample preparation unit and the incubator and the sample preparation unit and the analysis unit. In some embodiments, the system can centrifuge the multiwell plate and aspirate supernatant. In some embodiments, the system can aliquot liquid into wells of the multiwell plate and resuspend pelleted material. In some embodiments, the multiwell plate comprises wells having different concentrations of various antimicrobial agents to facilitate susceptibility analysis. In some embodiments, the system first identifies the infectious agent in the sample and optionally subsequently subjects the sample to antibiotic susceptibility testing. The system may be configured to minimize the number of wells analyzed by the flow cytometer based on identification of the infectious agent and prioritization of wells analyzed.

In some embodiments, the sample is a urine sample, a blood sample, a CSF sample, a respiratory swap sample, a nasal swab sample, an abscess sample, an ascites sample, a cyst sample, a Lavine swab sample, a sepsis sample.

In some embodiments, the system further comprises one or more buffers. In some embodiments, the system further comprises one or more growth mediums. In some embodiments, the system further comprises one or more centrifuges.

In some embodiments, the system further comprises a plurality of binding molecules, the binding moieties being conjugated with a detectable moiety. In some embodiments, the binding molecules are antibodies or binding fragments. In some embodiments, the detectable moiety is a dye (e.g., fluorescent dye, label) or other label. In some embodiments, the system allows for detection of at least 20 different fluorescent dyes.

In some embodiments, the system is organized into a sample preparation unit for accepting the sample, an incubation unit, and an analysis unit, the analysis unit comprising the flow cytometry unit, wherein the sample can move between both the incubation unit and the sample processing unit, and the sample can move between the analysis unit and the sample processing unit.

In some embodiments, the system comprises one or more computer processing units. In some embodiments, one or more of the computer processing units are communicatively connected (e.g., operatively connected) to the sample processing unit, the incubation unit, the analysis unit, or a combination thereof. In some embodiments, the computer processing unit comprises a microprocessor and one or more memory components, wherein at least one of the memory components comprises computer readable instructions for the microprocessor. In some embodiments, the computer readable instructions allow the microprocessor to regulate movement of the sample within or between the sample processing unit, the incubation unit, and analysis unit. For example, in some embodiments, the microprocessor may regulate movement of the sample within or between the sample processing unit, the incubation unit, and analysis unit by controlling a robotic arm. In some embodiments, the computer processing unit is configured to analyze results from the flow cytometer so as to provide identity of one or more infectious agents in the sample. In some embodiments, the one or more of the computer processing units are communicatively connected to the sample processing unit, the incubation unit, the analysis unit, or a combination thereof via a wire connection. In other embodiments, the one or more of the computer processing units are communicatively connected to the sample processing unit, the incubation unit, the analysis unit, or a combination thereof via a wireless connection. The processor and/or microprocessor is configured to execute the application and/or computer readable instructions.

As previously discussed, the systems and methods herein may also feature quantification of infectious agents, identification of the presence of an active infection, determining if infectious agents are alive, etc. The systems and methods herein may also have the ability to perform and/or integrate antibiotic susceptibility testing, sample preparation, sample processing, incubation, the like, or a combination thereof.

As an example, the system comprises an analysis unit, e.g., a flow cytometry unit or the component of the system that identifies (or allows for identification of) one or more infectious agents in the sample, quantifies (or allows for the quantification of) the infectious agents in the sample, identifies (or allows for identification of) one or more markers on or in the infectious agents in the sample, etc. The analysis unit or flow cytometry unit may feature an integrated processing system for analyzing data (e.g., from the flow cytometer) and providing results such as the identity of the one or more infectious agents, the number of the one or more infectious agents, the identity of the one or more markers, etc.

In addition to the analysis unit, the system may further comprise a preparation unit, e.g., a component of the system that prepares a sample for the analysis unit, e.g., flow cytometry analysis. In some embodiments, the system further comprises an incubation unit, e.g., a component of the system that may function to store, hold, retain, or promote the growth of the sample (e.g., infectious agents in the sample). The preparation unit and/or analysis unit and/or incubation unit may be operatively linked, e.g., wherein a sample can be moved from one unit to another. As an example, a sample may be inserted into the preparation unit. From the preparation unit, the sample may move to the analysis unit. In some embodiments, the sample may move from the preparation unit to the incubation unit. In certain embodiments, the sample moves from the incubation unit back to the preparation unit. In some embodiments, the sample may move from the analysis unit back to the preparation unit. In some embodiments, the system is a fully automated system.

As is described below, the system may further comprise one or more buffers, one or more growth mediums, one or more centrifuges, one or more waste containers, one or more incubators, etc. The system may comprise a magnetic separation rack if the system utilizes isolation using magnetic bead separation techniques. The system may further comprise (e.g., store) one or more antibodies conjugated with detectable dyes or labels, e.g., fluorescent labels. The system may also feature components for storing one or more reagents, components for measuring and/or dispensing one or more reagents to the sample, components for transporting the sample or a portion thereof from one unit to another unit, and/or components for transporting the sample or a portion thereof from one location to another within a unit. Such components for sample transportation, storing of buffers and reagents, dispensing buffers and reagents, etc. are well known to one of ordinary skill in the art.

The system further comprises processing units, memory units, and computer-implemented instructions for carrying out the methods herein and computer-implemented instructions for analyzing signals obtained from the analyzing unit.

For clarity, m refers to the total number of fluorophores used; n refers to the total number of targets to be identified (and thus also the total number of antibodies (antibody species)); l refers to the total number of unique antibody conjugates and l is greater than or equal to n; r refers to the number of fluorophores in a shared set of fluorophores and r is less than or equal to m; k is an index and represents an instance of a unique subset of the set of r (potentially up to m) fluorophores used; and i is a general index typically used for instances of targets. As an example, if the whole set of m fluorophores can be used to uniquely label all n antibody (no constraints on labeling other than each grouping is unique) m is at its minimum and l is likely at its maximum (most number of unique antibody fluorophore conjugates). If r only equals 1 for any subset, then m=n and all fluorophores are tied only to one antibody and all targets will only have one dominant fluorophore.

Any feature or combination of features described herein is included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### TERMS

Unless otherwise explained, all technical and scientific terms used herein have the same meaning as commonly understood by one of ordinary skill in the art to which a disclosed invention belongs. The singular terms “a,” “an,” and “the” include plural referents unless context clearly indicates otherwise. Similarly, the word “or” is intended to include “and” unless the context clearly indicates otherwise. The term “comprising” means that other elements can also be present in addition to the defined elements presented. The use of “comprising” indicates inclusion rather than limitation. Stated another way, the term “comprising” means “including principally, but not necessary solely”. Furthermore, variation of the word “comprising”, such as “comprise” and “comprises”, have correspondingly the same meanings. In one respect, the technology described herein related to the herein described compositions, methods, and respective component(s) thereof, as essential to the invention, yet open to the inclusion of unspecified elements, essential or not (“comprising”).

All embodiments disclosed herein can be combined with other embodiments unless the context clearly dictates otherwise.

Methods and materials that may be suitable for the practice and/or testing of embodiments of the disclosure are described below. Such methods and materials are illustrative only and are not intended to be limiting. Other methods and materials similar or equivalent to those described herein can be used. For example, conventional methods well known in the art to which the disclosure pertains are described in various general and more specific references, including, for example, Sambrook et al., Molecular Cloning: A Laboratory Manual, 2d ed., Cold Spring Harbor Laboratory Press, 1989; Sambrook et al., Molecular Cloning: A Laboratory Manual, 3d ed., Cold Spring Harbor Press, 2001; Ausubel et al., Current Protocols in Molecular Biology, Greene Publishing Associates, 1992 (and Supplements to 2000); Ausubel et al., Short Protocols in Molecular Biology: A Compendium of Methods from Current Protocols in Molecular Biology, 4th ed., Wiley & Sons, 1999; Harlow and Lane, Antibodies: A Laboratory Manual, Cold Spring Harbor Laboratory Press, 1990; and Harlow and Lane, Using Antibodies: A Laboratory Manual, Cold Spring Harbor Laboratory Press, 1999, Gene Expression Technology (Methods in Enzymology, Vol. 185, edited by D. Goeddel, 1991. Academic Press, San Diego, Calif.), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, Calif.), Culture of Animal Cells: A Manual of Basic Technique, 2^{nd} Ed. (R. I. Freshney. 1987. Liss, Inc. New York, N.Y.), Gene Transfer and Expression Protocols, pp. 109-128, ed. E. J. Murray, The Humana Press Inc., Clifton, N.J.), and the Ambion 1998 Catalog (Ambion, Austin, Tex.), the disclosures of which are incorporated in their entirety herein by reference.

All publications, patent applications, patents, and other references mentioned herein are incorporated by reference in their entirety for all purposes. In case of conflict, the present specification, including explanations of terms, will control.

Although methods and materials similar or equivalent to those described herein can be used to practice or test the disclosed technology, suitable methods and materials are described below. The materials, methods, and examples are illustrative only and not intended to be limiting.

In order to facilitate review of the various embodiments of the disclosure, the following explanations of specific terms are provided:

Flow cytometry: The present disclosure generally pertains to various embodiments of flow cytometry-based systems and methods. Basic information on flow cytometry can be found in numerous references, such as Shapiro's Practical Flow Cytometry, Third Edition (Alan R. Liss, Inc. 1995), which is incorporated by reference herein in its entirety. Although the basic principles of flow cytometry are known to those of ordinary skill in the art, Applicants believe that the embodiments presented in the present disclosure are currently unknown in the art.

Flow cytometry can be used to measure one or more optical or electrical parameters of cells or other particles that pass through a light beam (e.g., a laser). Generally, a fluid sample to be analyzed is introduced from a sample tube into or near the center of a faster flowing stream of sheath fluid, which carries the fluid sample toward the center of the combined streams, hydraulically compressing the sample and causing the cells or other particles in the sample volume to columnate. This process allows the cells or other particles to be delivered to the center of the measuring point in an examination zone. A laser beam is focused on the cells or other particles as they pass through the examination zone. Detectors that are optically connected to the examination zone interrogate signals from this zone on one or more detection channels. There may be one or more examination zones, illuminated by different lasers, separated spatially along he sample flow path. This spatial separation allows for temporal separation of the detected signals. When a cell or other particles in the flow stream is struck by one or more laser beams, certain signals are generated and sensed by detectors. The detectors utilize a plurality of detection channels. For instance, these signals include forward scatter intensity, which provides information concerning the size of individual cells or other particles. Another common signal is side scatter intensity, which provides information regarding the granularity (relative size, proportions, and refractive properties) of the cells or other particles. Other signals can include fluorescence emissions from one or more fluorescent dyes and/or fluorescent molecules associated with cells or other particles. Flow cytometers may include spectral flow cytometers having many more detection channels for different spectral emissions bands enabling a much larger number of unique fluorescent measurements than the number of fluorescent labels used to identify particles or cells, or other flow cytometer configurations, which are well known to one of ordinary skill in the art.

Flow cytometry systems typically feature fluidics systems, optics systems, and electronics systems. The fluidics system of a flow cytometer is responsible for transporting samples from the sample tube to the flow cell. Once through the flow cell (and past the laser), the sample is either sorted (in the case of cell sorters) or transported to waste. The components of the optical system may include excitation light sources, lenses, and filters used to collect and move the light around the instrument and the detection system that generates the photocurrent and/or counts photons. This information can be acted on in real time and/or saved for further analysis.

In spectral flow cytometry systems having a larger number of unique fluorescent measurements than conventional flow systems an unmixing algorithm is used to determine how much of each fluorescent label contributed to the aggregate measurements made across all fluorescent detectors. These algorithms are typically numerical minimizing the residual error for an objective function that relates the abundances of fluorophores to the measured optical outputs in an overdetermined system of linear equations which may also be subject to some form of measurement noise model as well as constraints limiting the solution set to physically possible values such as abundances must be positive and if expressed as fractions must sum to 1 [see Novo, Gregori and Rajwa]. Autofluorescence can complicate this analysis especially when it is variable across the various cells or particles being analyzed. When it is relatively consistent in form (the spectra) it can be effectively treated as an additional fluorophore in the numerical analysis. The advantage of spectral flow is its ability to make finer distinctions between emitters so that a larger set of labels can be used and distinguished in a single analysis. In many applications labels are created by conjugating fluorophores with antibodies specific to target antigens that may appear on the particles or cells to be analyzed. Appropriate fluorophore-antibody pairing can be used to advantage in certain defined applications.

As used herein, the term “antibody” or “antibodies” may refer to an antibody, a fragment thereof, or other binding moiety. As used herein, an “antibody fragment” refers to a partial or truncated version of an antibody that retains the ability to specifically bind to its target antigen. Non-limiting examples of antibody fragments include, but are not limited to, Fab, F(ab′)2, Fab′, Fv, scFv, sdAb, VHH, and dAb fragments.

As used herein, the term “primary antibody” refers to an antibody that directly binds the target of interest, and a “secondary antibody” refers to an antibody(s) that binds the primary antibody to amplify a signal.

As used herein, Degree of Labeling (DOL) refers to how many fluorophores/drugs/other molecules are conjugated to an antibody or fragment thereof.

The term infectious agent refers to an organism, agent, or entity that produces an infection. Infectious agents may include but are not limited to bacteria, viruses, fungi, protozoa, bacteria, or a combination thereof. Non-limiting examples of infectious agents that may be tested for in the present invention include but are not limited to one or a combination of: Acinetobacter baumannii, Actinotignum schaalii, Aerococcus urinae, Aerococcus urinae, Alloscardovia omnicolens, Candida albicans, Candida glabrata, Candida parapsilosis, Gardnerella vaginalis, Candida tropicalis, Chlamydia, Citrobacter freundii, Citrobacter koseri, Clostridium difficile, Corynebacterium riegelii, Klebsiella aerogenes, Enterococcus faecalis, Escherichia coli, Klebsiella oxytoca, Klebsiella pneumoniae, Morganella morganii, Mycobacterium tuberculosis, Mycoplasma genitalium, Mycoplasma hominis, Neisseria gonorrhea, Pantoea agglomerans, Proteus mirabilis, Providencia stuartii, Pseudomonas aeruginosa, Serratia marcescens, Staphylococcus aureus, coagulase-negative Staphylococcus, Streptococcus agalactiae, Streptococcus pyogenes, Viridans Group Streptococcus, Trichomonas vaginalis, Ureaplasma urealyticum, HHV-6, HHV-7, BK Virus, JC Virus, HSV 1&2, Adenovirus, CMV, Acinetobacter baumannii, Actinobaculum schaalii, Aerococcus urinae, Alloscardovia Omnicolens, Bacterioides species, BK Virus, Candida albicans, Candida auris, Candida glabrata, Candida parapsilosis, Citrobacter freundii, Citrobacter koseri, Clostridium species, Clostridium septicum, Coagulase Negative Staphylococcus (e.g., S. saprophyticus, S. epidermidis, S. lugdunensis, S. haemolyticus group), Corynebacterium group (e.g., Corynebacterium riegelii), Enterobacter group (e.g., E. aerogenes, E. cloacae), Enterococcus group (e.g., Enterococcus faecalis, Enterococcus faecium), Escherichia coli, Finegoldia magna, Human Herpes Virus -1,-2 (HSV -1,-2), Human Herpes Virus -5 (CMV), Human Herpes Virus -6 (HHV-6), JC Virus, Kingella kingae, Klebsiella group (e.g., Klebsiella oxytoca, Klebsiella pneumoniae), Morganella morganii, Mycobacterium group (e.g., Mycobacterium tuberculosis), Mycoplasma hominis, Pantoea agglomerans, Peptostreptococcus group (e.g., Peptostreptococcus prevotii, Peptostreptococcus micros, Peptostreptococcus tetradius, etc.), Peptoniphilus group, Porphyromonas group, Prevotella species, Proteus group (e.g., Proteus mirabilis), Providencia stuartii, Pseudomonas aeruginosa, Serratia marcescens, Staphylococcus aureus, Streptococcus agalactiae, Streptococcus pyogenes, Ureaplasma urealyticum, Viridans Group Strep (e.g., Streptococcus anginosus, Streptococcus oralis, Streptococcus pasteuranus group), Brucella species, Leptosphaerulina chartarum, Stenotrophomonas maltophilia, Salmonella typhi, Cryptococcus neoformans, Alternaria species, Aspergillus group, Aureobasidium pullulans, Candida auris, Candida group, Cladosporium herbarum, Exserohilum rostratum, Fusarium group, Humicola fuscoatra, Kodamaea ohmeri, Malassezia group, Phoma betae, Rhodosporidium species, Trichophyton mentagrophytes, Trichosporon species, Ulocladium botrytis, Porphyromonas asaccharolytica, etc.

The present invention is not limited to the infectious agents disclosed herein.

The terms antibiotic, antimicrobial agent and antibacterial agent refer to a compound or molecule for the purpose of killing or inhibiting the growth of an infectious agent. Antibiotics may herein be referred to as antimicrobial agents and/or anti-bacterial agents, or vice versa. Examples of antibiotics include, but are not limited to, penicillins, tetracyclines, cephalosporins, quinolones, lincomycins, macrolides, sulfonamides, glycopeptide antibiotics, aminoglycosides, carbapenems, ansamycins, lipopeptides, monobactams, nitrofurans, oxazolidinones, and polypeptides.

Penicillin antibiotics include, but are not limited to, penicillin, methicillin, amoxicillin, ampicillin, flucloxacillin, penicillin G, penicillin V, carbenicillin, piperacillin, ticarcillin, oxacillin, dicloxacillin, azlocillin, cloxacillin, mezlocillin, temocillin, and nafcillin. Additionally, penicillin antibiotics are often used in combination with beta-lactamase inhibitors to provide broader spectrum activity; these combination antibiotics include amoxicillin/clavulanate, ampicillin/sulbactam, piperacillin/tazobactam, and clavulanate/ticarcillin. Tetracycline antibiotics include, but are not limited to, tetracycline, doxycycline, demeclocycline, minocycline, and oxytetracycline. Cephalosporin antibiotics include, but are not limited to, cefadroxil, cephradine, cefazolin, cephalexin, cefepime, ceftaroline, loracarbef, cefotetan, cefuroxime, cefprozil, cefoxitin, cefaclor, ceftibuten, ceftriaxone, cefotaxime, cefpodoxime, cefdinir, cefixime, cefditoren, ceftizoxime, cefoperazone, cefalotin, cefamandole, ceftaroline fosamil, ceftobiprole, and ceftazidime. Cephalosporin antibiotics are often used in combination with beta-lactamase inhibitors to provide broader spectrum activity; these combination antibiotics include, but are not limited to, avibactam/ceftazidime and ceftolozane/tazobactam. Quinolone antibiotics include, but are not limited to, lomefloxacin, ofloxacin, norfloxacin, gatifloxacin, ciprofloxacin, moxifloxacin, levofloxacin, gemifloxacin, cinoxacin, nalidixic acid, trovafloxacin, enoxacin, grepafloxacin, temafloxacin, and sparfloxacin. Lincomycin antibiotics include, but are not limited to, clindamycin and lincomycin. Macrolide antibiotics include, but are not limited to, azithromycin, clarithromycin, erythromycin, telithromycin, dirithromycin, roxithromycin, troleandomycin, spiramycin, and fidaxomicin. Sulfonamide antibiotics include, but are not limited to, sulfamethoxazole, sulfasalazine, mafenide, sulfacetamide, sulfadiazine, silver sulfadiazine, sulfadimethoxine, sulfanilamide, sulfisoxazole, sulfonamidochrysoidine, and sulfisoxazole. Sulfonamide antibiotics are often used in combination with trimethoprim to improve bactericidal activity. Glycopeptide antibiotics include, but are not limited to, dalbavancin, oritavancin, telavancin, teicoplanin, and vancomycin. Aminoglycoside antibiotics include, but are not limited to, paromomycin, tobramycin, gentamicin, amikacin, kanamycin, neomycin, netilmicin, streptomycin, and spectinomycin. Carbapenem antibiotics include, but are not limited to, imipenem, meropenem, doripenem, ertapenem, and imipenem/cilastatin. Ansamycin antibiotics include, but are not limited to, geldanamycin, herbimycin, and rifaximin. Lipopeptide antibiotics include, but are not limited to, daptomycin. Monobactam antibiotics include, but are not limited to, aztreonam. Nitrofuran antibiotics include, but are not limited to furazolidone and nitrofurantoin. Oxaxolidinone antibiotics include, but are not limited to, linezolid, posizolid, radezolid, and torezolid. Polypeptide antibiotics include, but are not limited to, bacitracin, colistin, and polymyxin B.

Other antibiotics which are not part of any of the above-mentioned groups include, but are not limited to, clofazimine, dapsone, capreomycin, cycloserine, ethambutol, ethionamide, isoniazid, pyrazinamide, rifampicin, rifabutin, rifapentine, streptomycin, arsphenamine, chloramphenicol, fosfomycin, fusidic acid, metronidazole, mupirocin, platensimycin, quinupristin/dalfopristin, thiamphenicol, tigecycline, tinidazole, and trimethoprim.

The present invention is not limited to the antimicrobial agents, e.g., antibiotics or other drugs, disclosed herein. The present invention is not limited to the infectious agents disclosed herein.

Additionally, the scope of the presently disclosed methods and systems encompasses the inclusion of antibiotics, antimicrobials, or antibacterial agents not yet known, or not yet approved by regulatory authorities. The present invention is not limited to the antibiotics, antimicrobials, or antibacterial agents disclosed herein.

The processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., an FPGA (field programmable gate array) or an ASIC (application-specific integrated circuit).

Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for performing actions in accordance with instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks.

However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few. Devices suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry.

Computers typically include known components, such as a processor, an operating system, system memory, memory storage devices, input-output controllers, input-output devices, and display devices. It will also be understood by those of ordinary skill in the relevant art that there are many possible configurations and components of a computer and may also include cache memory, a data backup unit, and many other devices. To provide for interaction with a user, embodiments of the subject matter described in this specification can be implemented on a computer having a display device, e.g., an LCD (liquid crystal display), LED (light emitting diode) display, or OLED (organic light emitting diode) display, for displaying information to the user.

Examples of input devices include a keyboard, cursor control devices (e.g., a mouse or a trackball), a microphone, a scanner, and so forth, wherein the user can provide input to the computer. Other kinds of devices can be used to provide for interaction with a user as well; for example, feedback provided to the user can be in any form of sensory feedback, e.g., visual feedback, auditory feedback, or tactile feedback; and input from the user can be received in any form, including acoustic, speech, or tactile input. Examples of output devices include a display device (e.g., a monitor or projector), speakers, a printer, a network card, and so forth. Display devices may include display devices that provide visual information, this information typically may be logically and/or physically organized as an array of pixels. In addition, a computer can interact with a user by sending documents to and receiving documents from a device that is used by the user; for example, by sending web pages to a web browser on a user's client device in response to requests received from the web browser.

An interface controller may also be included that may comprise any of a variety of known or future software programs for providing input and output interfaces. For example, interfaces may include what are generally referred to as “Graphical User Interfaces” (often referred to as GUI's) that provide one or more graphical representations to a user. Interfaces are typically enabled to accept user inputs using means of selection or input known to those of ordinary skill in the related art. In some implementations, the interface may be a touch screen that can be used to display information and receive input from a user. In the same or alternative embodiments, applications on a computer may employ an interface that includes what are referred to as “command line interfaces” (often referred to as CLI's). CLI's typically provide a text based interaction between an application and a user. Typically, command line interfaces present output and receive input as lines of text through display devices. For example, some implementations may include what are referred to as a “shell” such as Unix Shells known to those of ordinary skill in the related art, or Microsoft® Windows Powershell that employs object-oriented type programming architectures such as the Microsoft® .NET framework.

Those of ordinary skill in the related art will appreciate that interfaces may include one or more GUI's, CLI's or a combination thereof. A processor may include a commercially available processor such as a Celeron, Core, or Pentium processor made by Intel Corporation®, a SPARC processor made by Sun Microsystems®, an Athlon, Sempron, Phenom, or Opteron processor made by AMD Corporation®, or it may be one of other processors that are or will become available. Some embodiments of a processor may include what is referred to as multi-core processor and/or be enabled to employ parallel processing technology in a single or multi-core configuration. For example, a multi-core architecture typically comprises two or more processor “execution cores”. In the present example, each execution core may perform as an independent processor that enables parallel execution of multiple threads. In addition, those of ordinary skill in the related field will appreciate that a processor may be configured in what is generally referred to as 32 or 64 bit architectures, or other architectural configurations now known or that may be developed in the future.

A processor typically executes an operating system, which may be, for example, a Windows type operating system from the Microsoft Corporation®; the Mac OS X operating system from Apple Computer Corp.®; a Unix® or Linux®-type operating system available from many vendors or what is referred to as an open source; another or a future operating system; or some combination thereof. An operating system interfaces with firmware and hardware in a well-known manner, and facilitates the processor in coordinating and executing the functions of various computer programs that may be written in a variety of programming languages. An operating system, typically in cooperation with a processor, coordinates and executes functions of the other components of a computer. An operating system also provides scheduling, input-output control, file and data management, memory management, and communication control and related services, all in accordance with known techniques.

Connecting components may be properly termed as computer-readable media. For example, if code or data is transmitted from a website, server, or other remote source using a coaxial cable, fiber optic cable, twisted pair, digital subscriber line (DSL), or wireless technology such as infrared, radio, or microwave signals, then the coaxial cable, fiber optic cable, twisted pair, DSL, or wireless technology are included in the definition of medium. Combinations of media are also included within the scope of computer-readable media.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIG. 1 shows a schematic view of the system of the present invention. The present invention is not limited to the components or configurations therein.

FIG. 2 shows a schematic view of the system of the present invention. The present invention is not limited to the components or configurations therein.

FIG. 3 shows an example of an ABX plate for antibiotic susceptibility testing (AST). The present invention is not limited to the plate configuration, the antimicrobial agents listed, or the concentrations thereof.

FIG. 4 shows a schematic view of methods and workflows utilizing the system of the present invention. The present invention is not limited to the component, configurations, methods, or workflows therein.

FIG. 5A shows a plan for testing specificity of antibodies against pooled organisms. The workup of how to produce such a pool is demonstrated.

FIG. 5B and 5C shows Klebsiella pneumoniae specificity. FIG. 5B shows the average RFU specificity of Klebsiella pneumoniae (KP) monoclonal antibodies (mAB) against Pool 1, Pool 2, and KP (as shown in FIG. 5A, single organism in the third flask being KP). FIG. 5C shows the normalized RFU specificity of KP monoclonal antibodies (mAB) against Pool 1, Pool 2, and KP. The y-axis denotes RFU.

FIG. 6A and 6B shows Enterococcus faecalis specificity. FIG. 6A shows raw specificity of Enterococcus faecalis (EF-2 (1:2)) against Pool 1, Pool 2, and EF only (as shown in FIG. 5A, single organism in the third flask being EF). FIG. 6B shows normalized specificity of EF-2 against Pool 1, Pool 2, and EF only. This ratio is the ratio of E. faecalis to a pool of organisms.

FIG. 7 shows theoretical arrangements of potential fluorophores using either three or five lasers. Specifically, the top image shows the potential of a 5 laser system and the bottom image shows the potential of the 3 laser system.

FIG. 8 shows potential interactions between fluorophores. Stained beads were used to demonstrate fluorophore profiles that may be used for spectral unmixing analysis. The unmixing analysis is the process utilized to decompose a spectrum into a collection of its constituent spectra. The abundances of each portion of the spectra can be used to determine the proportion of each fluorophore compared to others.

FIG. 9 shows a listing of fluorophores that are optimal for single and tandem use in this system. Using tandem fluorophores is one way to “barcode” the organisms and gain more information and have more combinations than using individual fluorophores.

FIG. 10 shows example overlays of fluorophore combinations that may be used to detect organisms. In this case, beads were used as a surrogate for different organisms to demonstrate this strategy.

FIG. 11 shows a sample 23 color panel design based on a flow cytometry machine (e.g., Cytek Aurora.).

FIG. 12 shows Escherichia coli commercial polyclonal antibodies against different E. colistrains. This demonstrates the potential variance in antibody success across different strains of a single organism. Despite potential reductions in specificity, discernible signals specific to the target organism were successfully identified when compared to negative controls.”

### DETAILED DESCRIPTION OF THE INVENTION

As previously discussed, the present invention features flow cytometry-based methods and systems for rapid identification of one or more target particles (e.g, cells or other particles, such as but not limited to infectious agents) in a sample. The methods and systems herein can quantify specific cells or particles in samples. In some embodiments, the methods and systems herein detect the presence of active infections. In some embodiments, the methods and systems herein determine antibiotic susceptibility of infectious agents to one or more antimicrobial agents (e.g., the pooled antibiotic susceptibility of the combination of the infectious agents to one or more antimicrobial agents) in the sample. The methods and systems herein help reduce or eliminate the need for empirical testing. The systems and methods herein may be fully automated, thereby eliminating the need for a user to perform steps after the raw sample or a preprocessed sample is introduced to the system.

In some embodiments, the infectious agent is a bacteria. In some embodiments, the infectious agent is a virus. In some embodiments, the infectious agent is a fungus. In some embodiments, the infectious agent is a parasite.

In some embodiments, the infectious agent is Acinetobacter baumannii, Actinotignum schaalii, Aerococcus urinae, Aerococcus urinae, Alloscardovia omnicolens, Candida albicans, Candida glabrata, Candida parapsilosis, Candida tropicalis, Chlamydia, Citrobacter freundii, Citrobacter koseri, Clostridium difficile, Corynebacterium riegelii, Klebsiella aerogenes, Enterococcus faecalis, Escherichia coli, Klebsiella oxytoca, Klebsiella pneumoniae, Morganella morganii, Mycobacterium tuberculosis, Mycoplasma genitalium, Mycoplasma hominis, Neisseria gonorrhoeae, Pantoea agglomerans, Proteus mirabilis, Providencia stuartii, Pseudomonas aeruginosa, Serratia marcescens, Staphylococcus aureus, Coagulase-negative Staphylococcus, Streptococcus agalactiae, Streptococcus pyogenes, Viridans Group Streptococcus, Trichomonas vaginalis, Ureaplasma urealyticum, HHV-6, HHV-7, BK Virus, JC Virus, HSV 1&2, Adenovirus, or CMV. The present invention is not limited to the aforementioned infectious agents.

The sample may be a biological sample, e.g., a sample derived from a patient or subject. For example, in certain embodiments, the sample is a urine sample, a blood sample, a CSF sample, a respiratory swap sample, a Levine swab, a nasal swab sample, an abscess sample, an ascites sample, a cyst sample, a sepsis sample, a biopsy, etc. The present invention is not limited to the aforementioned examples of samples.

The methods herein may feature sample preparation prior to flow cytometry analysis. In some embodiments, the sample (e.g., a portion of the sample) is centrifuged and ultimately resuspended and stained, e.g., incubated with a pool of antibody-fluorophore conjugates specific for a panel of targets, e.g., infectious agents. The sample may be centrifuged and washed one or multiple times after incubation with the pool of antibody-fluorophore conjugates prior to flow cytometry analysis. The present invention is not limited to preparation of the sample using centrifugation and washes. For example, in some embodiments, the system uses magnetic bead separation techniques.

In applications for identifying infectious agents in a sample, a portion of the sample may be used for antibiotic susceptibility testing (AST). The methods and systems herein may feature sample preparation prior to inoculation of the ABX plate for antibiotic susceptibility testing (AST). For example, a portion of the sample may be prepared (e.g., centrifuged and washed, subjected to magnetic bead separation techniques, etc.) and/or incubated in growth media prior to inoculation of the ABX plate.

### Antibodies

The present invention features antibodies, fragments thereof, or other binding moieties used for identifying targets, such as pathogens, in a sample. The present invention includes pools (or panels) of antibodies, fragments thereof, or other binding moieties used for identifying and distinguishing between pathogens.

The antibodies, fragments, or other binding moieties described herein may be labeled with a label (e.g., fluorophore) so as to be detectable in applications including but not limited to flow cytometry (e.g., manual and/or automated methods). The antibodies, fragments, or other binding moieties may be labeled in a way that allows for accurate distinction between multiple targets in a sample. For example, an antibody-label conjugate may have an emission spectra that distinguishes it from other similar fluorophore spectra in the analysis.

The present invention is not limited to use with pathogens. The present invention is not limited to use with flow cytometry. The present invention is not limited to fluorophore labels.

Without wishing to limit the present invention to any theory or mechanism, it is believed that the present invention is advantageous over other attempts to identify pathogens in samples using antibodies. Other systems are not designed to have antibodies with as high specificity. Others have not been able to distinguish between many pathogens in a polymicrobial sample.

In some embodiments, the pool of antibodies comprises at least 5 antibodies. In some embodiments, the pool of antibodies comprises at least 6 antibodies. In some embodiments, the pool of antibodies comprises at least 7 antibodies. In some embodiments, the pool of antibodies comprises at least 8 antibodies. In some embodiments, the pool of antibodies comprises at least 9 antibodies. In some embodiments, the pool of antibodies comprises at least 5 antibodies. In some embodiments, the pool of antibodies comprises at least 10 antibodies. In some embodiments, the pool of antibodies comprises at least 12 antibodies. In some embodiments, the pool of antibodies comprises at least 15 antibodies.

In some embodiments, one antibody is used to detect multiple strains of a particular pathogen (e.g., a species). In some embodiments, distinct antibodies are used to detect different strains of a particular pathogen (e.g., a species). In some embodiments, the same label (e.g., fluorophore) is used with the different antibodies that each detect a different strain of a pathogen. In some embodiments, different labels (e.g., fluorophores) are used with different antibodies.

In some embodiments, a single antibody is capable of detecting all or most strains of a species and thus there is one fluorophore required. In other embodiments, multiple antibodies may be required to provide coverage and detect all strains of a species. In this case, the antibodies used would be labeled with the same fluorophore so they may be detected together as one species.

In some embodiments, one antibody is used to detect multiple infectious agents (e.g., multiple pathogens including but not limited to bacteria, yeast, viruses, or parasites). In some embodiments, distinct antibodies are used to detect different infectious agents (e.g., multiple pathogens including but not limited to bacteria, yeast, viruses, or parasites). In some embodiments, the same label (e.g., fluorophore) is used with the different antibodies that each detect different infectious agents. In some embodiments, different labels (e.g., fluorophores) are used with different antibodies.

### Data Analysis System

The methods and systems herein feature a data analysis method that utilizes fluorophore-labeled antibodies (antibody-fluorophore conjugates) specific to antigens located on or in target particles (e.g., cells or other particles, such as but not limited to infectious agents) which appear in abundance preferentially on each one of the various target particles of interest (e.g., cells or other particles of interest). As used herein, the term “target particle” refers to a target cell or other target particle. An antibody (thus also an antibody-fluorophore conjugate) can be created to bind preferentially, e.g., via specificity for antigen on or in the target cell or target particle.

The present invention describes spectral flow cytometry and the use of spectral flow cytometers. Non-limiting examples of spectral flow cytometers include Cytek Aurora, SONY SA3800, and Invitrogen Bigfoot. See also U.S. Pat. No. 7.280,204, U.S. Pat. App. No. 2013/0346023, U.S. Pat. No. 9,128,955, and Novo et al., Cytometry 2013 83(5): 508-520, the disclosures of which are incorporated in their entirety herein by reference.

The present invention includes methods for selecting fluorophores for particular antibodies to prepare the antibody-fluorophore conjugates. For example, antibody-fluorophore conjugates may be prepared based on a variety of criteria which, among others, may include: the known probabilities that various target particles (e.g., infectious agents) will appear together in sample (e.g., a patient sample), the similarities and differences between the fluorophores to be selected from, the degree of specificity of the antibody for its target particle, the degree of non-specific binding of the antibody on the various non-target particles or other non-targets, the intensity of the fluorophore, and/or the intensity and the spectra of the autofluorescence associated with the target particle and/or its target antibody.

In preparation for flow cytometry, a portion of the sample is incubated with the appropriate pool of antibody-fluorophore conjugates. In some embodiments, a single portion of the sample is incubated with all of the desired antibody-fluorophore conjugates to be used in the assay. In some embodiments, for example, if the sample is being tested for more targets than the flow cytometer can detect in a single run, the antibody-fluorophores are split into two or more groups, wherein a portion (e.g, a first portion) of the sample is incubated with the first group of antibody-fluorophore conjugates, and a portion (e.g., a second portion) of the sample is incubated with the second pool of antibody fluorophore conjugates. The present invention is not limited to splitting the antibody-fluorophore conjugates into two groups. In some embodiments, the antibody-fluorophore conjugates are split into three groups for three flow cytometry runs. In some embodiments, the antibody-fluorophore conjugates are split into four groups for four flow cytometry runs, etc.

If the overall particle or cell concentration of the sample is not known, it may be beneficial to prepare dilutions of the sample, e.g., where the goal would be to prepare a dilution that has a concentration that will be acceptable for flow cytometric analysis. In some embodiments, two dilutions are prepared for each group of antibody-fluorophore conjugates. In some embodiments, three dilutions are prepared for each group of antibody-fluorophore conjugates. The present invention is not limited to three dilutions. As an example, three wells are prepared for each group of antibody-fluorophore conjugates. The first well may reflect the concentration that is nominally the original sample concentration, and the second two wells may be, for example, 1/10 and 1/100 the original sample concentration.

In some embodiments, the methods feature initial measurements to assess the cell concentration prior to automatic dilution, and then the sample can be diluted (or not) accordingly to optimize analysis on the cytometer.

Antibodies are selected for a given type of target particle (e.g., cell or other particle) to a target antigen specific to that target particle (e.g., cell or other particle) type. In the case of a biological species, the antibodies are typically specific to target antigens that may be conserved across multiple variants of that species and for which there is minimal binding (specific or otherwise) with the other particles of interest in the analysis.

Fluorophores may be selected such that they can each be differentiated via spectral flow cytometry. In some embodiments, each target particle is intended to be identified with a unique fluorophore, however, the present invention is not limited to this configuration. As will be described further below, the present invention also includes the identification of a number of targets with a fewer number of different fluorophores than the number of targets by utilizing combinations of sets of fluorophores.

In some embodiments, the fluorophores may be paired (conjugated) with the various antibodies based on a set of criteria in order to augment differentiation for each event and normalize the intensity of the target fluorophore for each target particle (e.g., cell or other particle). The criteria may include one or the following: the degree of labeling (DOL) of the fluorophores onto the conjugated antibodies, which may be kept relatively low to preserve the binding specificity of the target antibody to the specific antigen of the target particle; the brightness of the fluorophore may generally be inversely proportional to binding selectivity of the target antibody versus those of the non-target antibodies for the particular target particle; the criteria described above (re: DOL of the fluorophores to the conjugated antibodies being kept relatively low) may be augmented by adjusting for antibody binding density on the target particle, for example, dimmer fluorophores may be conjugated with target antibodies with higher binding densities to normalize overall intensity of the event; and/or autofluorescence of the target particle or its target antibody may be used to augment differences between similar fluorophores so that a target particle or antibody with a distinctive and significant autofluorescence spectra may have its target antibody conjugated with a fluorophore with distinctly different spectra than the autofluorescence of the target particle but which also has a relatively similar spectra to another fluorophore that will be utilized for a different antibody targeting another type of particle. In other words, factors that may affect which fluorophore is chosen for a particular antibody may include the autofluorescence signature of the target and/or its target antibody, the brightness or dimness of the fluorophore, the abundance of the corresponding antigen on the target particle, the growth curve of the target particle (e.g., cell). The fluorophore is generally chosen to help enhance resolution of the possible target particles.

As will be described herein, the identification of the target particle (e.g., cell or other particle) is primarily based on the identification of the dominantly abundant antibody-fluorophore conjugate on the target particle.

The flow cytometer features an analysis system configured to utilize an unmixing algorithm that incorporates an additional constraint on the acceptable solution set, e.g., a minimum abundance ratio requirement. This constraint may (in part) be determined by the relative specificity of a given conjugate for the specific antigen on its target particle. For example, the constraint may be that the solution must be such that the abundance of the most highly abundant fluorophore is greater than X times the next most abundant fluorophore in order for its conjugate antibody's targeted particle to be a candidate solution. X refers to the minimum abundance ratio and is associated with the antibody. The lowest bar for this is that the dominant fluorophore for any single event has an abundance that is greater than X times any of the other fluorophore abundances wherein X is at least greater than 1, e.g., a minimum abundance ratio requirement wherein X is a single value representing the minimum realized selectivity ratio across all of the fluorophore conjugates when binding to their respective target particle. The value of X does not have to be precise; it needs to be large enough to eliminate ambiguous solutions due to noise and unaccounted for autofluorescence while being small enough to allow for the existence of likely amounts of a non-target binding as can be measured a priori, e.g., by exposing a given target particle to a cocktail of all (l) antibody conjugates for n target particles of interest to determine the appropriate value to assign.

In some embodiments, this constraint can be employed in n instances of the numerical optimization, wherein n equals the number of different antibodies, which is typically equal to the number of potential target particles (e.g., infectious agents) to be identified. For example, in an experiment involving 20 different possible target particles and 20 different antibody-fluorophore conjugates, each specific for a particular target particle (e.g., a distinct infectious agent), the constraint can be employed uniquely in 20 instances of the numerical optimization. As previously discussed, X may be dependent on the conjugate's (antibody's) relative selectivity for its target particle. Based on prior measurements, the system can assign a particular value for X for each antibody-fluorophore conjugate representing its antibody conjugate binding selectivity. For example, for a system that utilizes unique fluorophores for each antibody species, the system can assign a value for X for each of: Fluorophore A (antibody-fluorophore conjugate A), Fluorophore B (antibody-fluorophore conjugate B), Fluorophore C (antibody-fluorophore conjugate C), Fluorophore D (antibody-fluorophore conjugate D), Fluorophore E (antibody-fluorophore conjugate E), Fluorophore F (antibody-fluorophore conjugate F), Fluorophore G (antibody-fluorophore conjugate G), Fluorophore H (antibody-fluorophore conjugate H), Fluorophore I (antibody-fluorophore conjugate I), Fluorophore J (antibody-fluorophore conjugate J), Fluorophore K (antibody-fluorophore conjugate K), Fluorophore L (antibody-fluorophore conjugate L), Fluorophore M (antibody-fluorophore conjugate M), Fluorophore N (antibody-fluorophore conjugate N), Fluorophore O (antibody-fluorophore conjugate O), Fluorophore P (antibody-fluorophore conjugate P), Fluorophore Q (antibody-fluorophore conjugate Q), Fluorophore R (antibody-fluorophore conjugate R), Fluorophore S (antibody-fluorophore conjugate S), Fluorophore T (antibody-fluorophore conjugate T), Fluorophore U (antibody-fluorophore conjugate U), Fluorophore V (antibody-fluorophore conjugate V), Fluorophore W (antibody-fluorophore conjugate W), Fluorophore X (antibody-fluorophore conjugate X), Fluorophore Y (antibody-fluorophore conjugate Y), and Fluorophore Z (antibody-fluorophore conjugate Z), or an array of X values of length n, wherein n is the number of unique targets to identify, which in this case equals the number of fluorophores to identify. This may be desirable when some X values approach 1 wherein the value may be considered generally too low to apply generally but acceptable to apply to individual conjugates that may typically not occur at anywhere near that relative abundance value unless the particle was actually the one for which it is targeted. This might be the case for a target particle wherein the antigen that is conserved across potentially different variants of the target particle is typically present in low density on the target particle; this may allow non-specific binding of other conjugates to become more comparable to the targeted binding. It is also possible that with the array of values being utilized in separate instances, a value could be less than 1 as long as it is distinct from the ratios that might exist on any target particle other than for the one for which it was designed. While it may not be considered optimal, it may be possible.

In some embodiments, each of Fluorophores A-Z have a unique value for X. In some embodiments, a portion of Fluorophores A-Z have a unique value for X. In some embodiments, a single X value can be utilized. In this case, the single value of X chosen would be the minimum of all of the A-Z X values.

In the case where a single X value can be utilized, it may be important that X is greater than 1 so that there is always a single dominant antibody-fluorophore conjugate. While X may be more likely to be on the order of 10 or more, there may be circumstances wherein X is less than 10, which can still allow the ability to uniquely identify a target particle. In the case where an X value for a given antibody-fluorophore conjugate approaches 1 (or for cases in which it is helpful to accommodate autofluorescence of some or all antibody conjugates), it may be beneficial to use separate instances of constraints on X and, in this case, X values can potentially be lower than in the single instance calculation. In some embodiments, X for a particular antibody-fluorophore conjugate may be less than 1. In some embodiments, X for a particular antibody-fluorophore conjugate may be 1 or about 1. In some embodiments, X for a particular antibody-fluorophore conjugate is 2 or about 2. In some embodiments, X for a particular antibody-fluorophore conjugate is 3 or about 3. In some embodiments, X for a particular antibody-fluorophore conjugate is 4 or about 4. In some embodiments, X for a particular antibody-fluorophore conjugate is 5 or about 5. In some embodiments, X for a particular antibody-fluorophore conjugate is 6 or about 6. In some embodiments, X for a particular antibody-fluorophore conjugate is 7 or about 7. In some embodiments, X for a particular antibody-fluorophore conjugate is 8 or about 8. In some embodiments, X for a particular antibody-fluorophore conjugate is 9 or about 9. In some embodiments, X for a particular antibody-fluorophore conjugate is 10 or about 10. In some embodiments, X for a particular antibody-fluorophore conjugate is more than 10, e.g., about 11, 12, 13, 14, 15, 16, 17, 18, 19, 20, etc. In some embodiments, X for a particular antibody-fluorophore conjugate is from 1 to 2. In some embodiments, X for a particular antibody-fluorophore conjugate is from 2 to 3. In some embodiments, X for a particular antibody-fluorophore conjugate is from 3 to 4. In some embodiments, X for a particular antibody-fluorophore conjugate is from 4 to 5. In some embodiments, X for a particular antibody-fluorophore conjugate is from 5 to 6. In some embodiments, X for a particular antibody-fluorophore conjugate is from 6 to 7. In some embodiments, X for a particular antibody-fluorophore conjugate is from 7 to 8. In some embodiments, X for a particular antibody-fluorophore conjugate is from 8 to 9. In some embodiments, X for a particular antibody-fluorophore conjugate is from 9 to 10. The present invention is not limited to the aforementioned values for X.

Knowing this relative selectivity of the various conjugates for their intended targets can help enhance the analysis process. For example, the system can be configured to help rule out false positives with the use of the known value of X that is appropriate in order to give a certain level of confidence in the result based on knowledge of the relative binding characteristics of the conjugates. In some embodiments, X can be different for each target conjugate so that the criteria represent an array of values relating to each possible target particle identification. In some embodiments, e.g., for a closed form solution, this can be applied after the fact to see if a valid identification has been made. In some embodiments, this can also be applied as a constraint in the numerical analysis, in which case the analysis could be done for each of n possible outcomes where n is the number of possible targets for identification. As a non-limiting example, to identify the particle as a Type 1 particle out of n possible targets, the measured abundance of a₁ in that instance (abeing associated with the assigned antibody-fluorophore conjugate for a Type 1 particle) must meet the constraint that it is greater than X₁ times any other abundance a through aₘ, wherein m is the number of all of the different fluorophores used for labeling in the assay and X₁ is an approximate predetermined value for X for the antibody for a Type 1 particle. This would be performed n times (n instances) and the solution with the minimum residual error represents the correct solution and provides the correct identity of the target particle (e.g., cell or other particle, e.g., infectious agent).

The unmixing of the spectra in spectral flow cytometry is different from the “compensation” algorithms in conventional flow because the problem is overdefined. Instead of solving a system of linear equations with a square matrix (equal number of fluorophores to the number of detected spectral measures), the problem is over-defined with more measures than fluorophore abundances to determine. There are many approaches in the literature to unmixing the spectra from a multi-fluorophore experiment on a spectral flow cytometer, but a dominant assumption is that autofluorescence is either ignored or is consistent (at least in spectral form) across the events and, as such, can even be treated as another fluorophore in the analysis. Further, a common addition to these algorithms is some assumption about the form of a noise model (which, if used in the absence of abundance constraints, can still lead to a closed form solution). A common constraint is the requirement that all abundances are positive, which leads to the requirement for a numerical solution but eliminates solutions that are physically impossible. Yet if all abundances are required to be greater than zero, many events get forced to the zero line on that fluorophore's axis which can be problematic when plotting results and/or interpreting binding ratios. However, based on the selection of fluorophores and conjugate selectivity described herein, this positivity requirement is likely unnecessary as any abundances near zero are not likely relevant.

It is possible that autofluorescence could be a large source of interference with the analysis since there are potentially different autofluorescence signatures for each (or at least a portion of) the different target types of interest (e.g., particles, cells, etc., e.g., in the case of different species of biological particles such as bacteria). In some embodiments, a mechanism for addressing the autofluorescence in the presence of particle types with different autofluorescence spectra is to premeasure (and store) the form of the autofluorescence spectra for each particle type individually.

The system may be configured to assign an autofluorescence signature (S) for each target particle (e.g., infectious agent). In some embodiments, the autofluorescence signatures (S) are each unique for each target (e.g., infectious agent); however, the present invention is not limited to each target having a unique fluorescence signature (S).

These autofluorescence spectra may be added as an additional fluorophore in the analysis with its own unknown abundance. As previously discussed, the closed form solutions could be used (e.g., one each for each of the n unique autofluorescence spectra), and then the solutions could be tested against the corresponding fluorescence abundance requirement Xᵢ for each target particle with the addition of autofluorescence spectra S having abundance aₘ₊₁ wherein m equals the number of fluorophores used for labeling n antibody species/target particles (note that m=n in the case of labeling each antibody species with a unique single fluorophore, but the present invention is not limited to each antibody being labeled with a single unique fluorophore). An optimization formulation requiring a numerical solution can be performed by the imposition of the abundance requirement (using Xᵢ) paired with the addition of the appropriate autofluorescence spectra Sᵢ as an additional fluorophore and use of an appropriate numerical approach to solve each of the n problems to determine the best fit. This approach can be coupled with elimination of many of the possible solutions by examination of the measured spectra using certain key measurements (e.g., distinct characteristics for each target fluorophore) to identify incompatibility with the minimum abundance ratio requirement immediately. This may take the form of identifying absolute intensities or threshold ratios of intensity at or between key measurements (spectral ranges excited by various lasers) for the given fluorophore, i, and noting when these are not sufficient for i to possibly be the dominant fluorophore. For example, if the spectra for fluorophore i has a peak intensity at a certain detected range (e.g., 704-736 nm when excited by a violet laser) and a relatively low intensity peak in this range is observed compared to the other detected intensities in the actual measurement, then i cannot be the dominant fluorophore resulting in that spectra. In many cases, a set of elimination criteria can be used in evaluating the observed spectral measurement to quickly rule out a portion of the n possible fluorophores. The higher the Xᵢ and/or the more unique the spectra is relative to the spectra of the other m possible fluorophores, the easier it would be to detect measured spectra that cannot include fluorophore i as the solution (the dominant fluorophore).

Without wishing to limit the present invention to any theory or mechanism, it is believed that the methods herein are advantageous because a different autofluorescence signature can be evaluated for each separate instance of the numerical optimization. This autofluorescence signature will be that of the corresponding target particle, antigen, cell, etc. (e.g., infectious agent) for the potential dominant fluorophore representing the target particle chosen in that iteration. These n instances can be executed in parallel with the one with the lowest residual error determining the identity of the particle, antigen, cell, etc. (e.g., infectious agent). If not executed in parallel, the analysis may be further reduced by evaluating key characteristics of the measured spectra to eliminate non-viable dominant fluorophores prior to unmixing.

Increasing the number of fluorophores that must be identified in a system increases the complexity of the system by requiring additional unique detectors and or lasers. Similar fluorophores become more common, and this can also require more care in calibration in measuring the controls used to establish each of the unique fluorophore spectra. In certain embodiments, the systems and methods herein require one unique fluorophore for each unique particle type to be identified. However, the present invention is not limited to this configuration. For example, as a means to help reduce the number of fluorophores that must be distinguished by the system (which can help minimize the cost of the system and simplify its operation) while enabling the same or a larger number of particle types to be identified, the concept of the minimum realized selectivity ratio can be used to select target antibodies that have very high values of X and provide an array of conjugates for each of these using a set of 2 or more different fluorophores. For example, the total number of distinct particles that could be identified with m fluorophores would be the sum of all the terms m!/((m/−k)!k!) for all k from 1 to m. In some embodiments, the number of particles that the system can identify using a subset of r fluorophores is the sum of each of the terms r!/((r−k)!*k!) for all k from 1 to r, wherein r can be less than or equal to m. For example, it may be possible to distinguish 3 targets with 2 fluorophores (Target 1=Fluorophore A, Target 2=Fluorophore B, and Target 3=½ Fluorophore A and ½ Fluorophore B); 7 targets with 3 fluorophores (Target 1=Fluorophore A, Target 2=Fluorophore B, Target 3=Fluorophore C, Target 4=½ Fluorophore A and ½ Fluorophore B, Target 5=½ Fluorophore A and ½ Fluorophore C, Target 6=½ Fluorophore B and ½ Fluorophore C, Target 7=⅓ Fluorophore A and ⅓ Fluorophore B and ⅓ Fluorophore C), 15 targets with 4 fluorophores, (Target 1=Fluorophore A, Target 2=Fluorophore B, Target 3=Fluorophore C, Target 4=Fluorophore D, Target 5=½ Fluorophore A and ½ Fluorophore B, Target 6=½ Fluorophore A and ½ Fluorophore C, Target 7=½ Fluorophore A and ½ Fluorophore D, Target 8=½ Fluorophore B and 1/2 Fluorophore C, Target 9=½ Fluorophore B and ½ Fluorophore D, Target 10=½ Fluorophore C and ½ luorophore D, Target 11=⅓ Fluorophore A and ⅓ Fluorophore B and ⅓ Fluorophore C, Target 12=⅓ Fluorophore A and ⅓ Fluorophore B and ⅓ Fluorophore D, Target 13=⅓ Fluorophore A and ⅓ Fluorophore C and ⅓ Fluorophore D, Target 14=⅓ Fluorophore B and ⅓ Fluorophore C and ⅓ Fluorophore D, Target 15=¼ Fluorophore A and ¼ Fluorophore B and 1/4 Fluorophore C and ¼ Fluorophore D), etc. For reference, U.S. Pat. No. 6,514,295 describes staining microspheres with two or more fluorescent dyes, the disclosure of which is incorporated in its entirety herein by reference.

In one embodiment, if using k fluorophores with k equal to at least 1 and less than or equal to r (wherein r is the number of a set of fluorophores used in various combinations to uniquely identify various targets) to identify a given target, a portion of the antibody for that target would be labeled separately by one each of the k fluorophores, and then these would be combined to provide the total number of antibodies that would have been used if labeled by a single fluorophore, e.g., the total conjugates for that antibody would be made up of a fractions of approximately 1/k of the total antibody separately labeled with one each of the k fluorophores. For example, if 3 fluorophores are used to identify Target A, then k=3, and ⅓ of the antibody for Target A is labeled with a first fluorophore, ⅓ of the antibody for Target A is labeled with a second fluorophore, and ⅓ of the antibody for Target A is labeled with a third fluorophore. The final stained target particle would have approximately 1/k (⅓ in the example of Target A) of each of these k fluorophores bound thereon in dominant abundance over any other fluorophores used to label conjugates and having an X value for each that is approximately ⅓ of X_{A} (wherein X_{A} is the minimum realized selectivity ratio of the single fluorophore labeled antibody for Target A). For a Target B, C, and D, separate subsets of 2 of the same set of 3 fluorophores can be used to separately label their corresponding antibodies, creating a target criteria with 2 fluorophores having dominant and roughly equal abundances over all other fluorophores used to stain targets in which the minimum realized selectivity ratio for the fluorophores in each pair is approximately ½ of the corresponding ratio, Xᵢ, for each of the single fluorophore labeled antibodies for Targets B, C and D respectively. Finally, 1 of each of the set of 3 fluorophores used for Target A can be used, one each, to label the corresponding antibodies for Targets E, F and G creating target criteria for identifying each which is approximately the same as it would have been if all antibodies were labeled with a single unique fluorophore.

Without wishing to limit the present invention to any theory or mechanics, it is believed that in order for this to be effective with the use of the abundance criteria, the original value of Xᵢ for a given antibody should be relatively high as the new value for a fluorophore on the target particle labeled with k fluorophores will be 1/k of the original and there will be additional non-specific binding of the same fluorophores used on other target particles (labeling other antibodies) which may reduce it further. To use this approach, for Target i with minimum abundance ratio Xᵢ (selectivity ratio when singly labeled), the minimum abundance ratios for each fluorophore when labeled with k fluorophores are each approximately Xᵢ/k and the minimum abundance ratio requirement would take the form of the conjugate requirement that a₁>Xᵢ/k AND a₂>Xᵢ/k . . . AND aₖ>Xᵢ/k). Each of the abundances of the k fluorophores would be about equal and must all be greater than Xᵢ/k times each of the other m−k fluorophores used to label other targets. Fluorophores may only be combined in this way for antibodies with relatively high minimum realized selectivity (large values of X) because of this dilution. In this way, it is possible to use only 4 fluorophores to distinguish 15 different target particles, and it is also possible, for example, to use two sets of 3 fluorophores to distinguish 7 targets each (totaling 14 targets detectable with the 2 sets of 3 fluorophores) plus another set of 6 fluorophores to be individually used to distinguish 6 additional targets with lower X values, yielding an assay that can detect 20 different targets distinguishable with a total of 12 fluorophores (without diluting the 6 with lowest X values or allowing 6 to be used uniquely in conjunction with significant target or target antibody autofluorescence, for example.

The system may be configured to ignore non-target particles, e.g., non-target cells, depending on the application, without interfering with the identification or counting of the target particles of interest (e.g., cells or other particles such as but not limited to infectious agents). For example, in some embodiments, such as an application for the identification of infectious agents, large cells (e.g., as detected from forward scatter) may be ignored or counted as non-target cells, such as red blood cells, white blood cells, etc.

The system of the present invention can also be physically tuned, e.g., aspects of the flow cytometer such as but not limited to the filters, laser intensities, detector gains, etc., can be adjusted and optimized for a particular set of fluorophores and Xᵢ values to optimize the system's ability to easily distinguish the various fluorophores and simplify the analysis and/or improve confidence in the results.

As previously discussed, the system may require a threshold level of fluorescence. There may be certain events wherein the threshold level of fluorescence is met and the size of the target particle (e.g., cell) is appropriate but there is not an appropriate significantly dominant fluorophore. For example, while the system requires a dominant fluorophore as a constraint, the residual error for the best case may be exceptionally high, meaning it is not a good fit. In the aforementioned event, the system may ignore the event (e.g., cell) completely and not count it as anything. As a non-limiting example, this may be possible for fastidious pathogens that require a special component in the growth media to grow that will not be labeled in the final antibiotic susceptibility testing (AST) analysis. This may also be possible for non-labeled infectious agents in general (e.g., fastidious in one set and non-fastidious in the other, for example if the sample and set of fluorophores are split into two groups or sets) in the identification analysis. This situation may also result from coincidence (e.g., multiple targets occupying the interrogation zone simultaneously) between multiple targets of different types which would yield a poor fit to the constraint of a single dominant fluorophore. This combination of error threshold, low fraction and low intensity can be calculated.

In some embodiments, if the total concentration of a cell type identified as a particular infectious agent is greater than a predetermined infection threshold, the sample is considered to be positive for that infectious agent. In some embodiments, if the total concentration of a cell type identified as a particular infectious agent is less than a predetermined infection threshold, the sample is considered to be negative for that infectious agent. The infection threshold may be an industry standard, a laboratory standard, etc. For example, a commonly used infection threshold is 10⁴ cells/ml; however, the present invention is not limited to this particular infection threshold. In some embodiments, if an infectious agent is below the infection threshold, the infectious agent may not necessarily be considered in further analysis.

The present invention is not limited to use with pathogens. The present invention is not limited to use with flow cytometry. The present invention is not limited to fluorophore labels.

### Susceptibility and Resistance Analysis for Infectious Agents

Upon detection and identification of one or a combination of (relevant) infectious agents, the system can next perform an antibiotic susceptibility testing (AST) analysis.

The system can utilize a database formed from historical data or data previously collected to predict the probability of susceptibility of the one or combination of infectious agents in the sample against various concentrations and/or various types of antimicrobial agents in an antibiotic susceptibility testing (AST) analysis, e.g., using an ABX plate, to determine which wells of the ABX plate are optimal to test, which ones are to be prioritized first, and which are likely unnecessary to analyze.

The system may feature a multiwell plate (ABX plate) wherein a plurality of the wells house various individual antimicrobial agents at various particular concentrations or a combination of antimicrobial agents at particular concentrations. For example, the ABX plate may feature a series of concentrations of one or more of the following antimicrobial agents: Ciprofloxacin, Levofloxacin, Tetracycline, Cefaclor, Nitrofurantoin, Ampicillin, Sulfamethoxazole/Trimethoprim, Ceftriaxone, Gentamicin, Cefazolin, Cefepime, Cefoxitin, Cefazidinme, Meropenem, and/or Vancomycin. In some embodiments, the ABX plate may feature a series of concentrations of one or more of the following combinations of antimicrobial agents: Piperacillin/Tazobactam, Amoxicillin/Clavulanate, and/or Ampicillin/Sulbactam. The present invention is not limited to the antimicrobial agents disclosed herein nor the concentrations thereof. Wells may also feature growth media disposed therein prior to inoculation with the sample. In some embodiments, a portion sample is prepared for AST analysis in growth media and the antimicrobial agents are inoculated with aliquots of sample-growth media.

In some embodiments, all of the wells (or all of the appropriate wells) are inoculated with a portion of the sample. In some embodiments, certain wells are not inoculated if, for example, it has been predetermined, based on the identification of infectious agents, that the particular antimicrobial agent and/or concentration thereof will be irrelevant to the analysis. After inoculation, the plate is then incubated for a nominal time which may be a function of the infectious agents identified in the prior step or may be a fixed nominal time.

After incubation, the plate is removed from the incubator. In some embodiments, the plate is centrifuged, and the supernatant is removed from each appropriate well. As previously discussed, the present invention is not limited to centrifugation techniques, and other preparatory techniques are within the scope of the present invention. In some embodiments, an antibody cocktail is added, which will have antibody conjugates for each pathogen of interest. The antibody cocktail may not feature antibody conjugates directed to fastidious organisms, e.g., organisms that would not be likely to grow during the incubation in the growth media. In some embodiments, the antibody cocktail is added only to the wells that have not been predetermined to be excluded from analysis. The plate may then be allowed to incubate for a period of time. After incubation, the plate may then be prepared, As a non-limiting example, the plate may be centrifuged, the supernatant removed from the appropriate wells, wells resuspended with a wash buffer, etc. This centrifuge and resuspension may be repeated multiple times, e.g., once, 2×, 3×, 4×, 5×, etc. In some embodiments, only the wells that have not been predetermined to be excluded from analysis will be prepared.

Next, the system will analyze the ABX plate one well at a time, e.g., via flow cytometry. The concentration of the various target infectious agents will be determined and assessed as to whether sufficient growth has occurred to conclude that the given pathogen is resistant to the components of that well. If not, it is susceptible. In this way, the system is configured to determine the antimicrobial agents and/or concentrations thereof to which the sample is susceptible and to which the sample is resistant. This allows the system to determine the minimum susceptible concentration of the relevant antimicrobial agents, e.g., the minimum inhibitory concentrations.

In certain embodiments, the system analyzes each of the wells seeded with the sample. In some embodiments, the system selects particular wells to analyze first. For example, the system may organize the wells in a particular order in which it intends to analyze based on one or more predictive criteria. In some embodiments, the results of one well may result in the changing of the order of the wells for the subsequent analysis. Some wells may not be analyzed based on the results of previously analyzed wells.

Without wishing to limit the present invention to any theory or mechanism, it is believed that the methods and systems of the present invention are advantageous because the methods and systems can incorporate the use of overall population probabilities and conditional probabilities of resistance (or susceptibility) at various concentrations against various antimicrobial agents.

A database containing measured resistances and minimum inhibitory concentrations (MICs) of samples of various infectious agents and combinations of infectious agents (polymicrobial infections) against various concentrations of various antimicrobial agents or combinations of antimicrobial agents can be used to determine the probabilities of resistance (p_{R}) for individual infectious agents when exposed to the various concentrations of the various antimicrobial agents. In some embodiments, the p_{R} can be used to select the order of well analysis that will result in the fewest wells needed to be analyzed in order to get the desired information, e.g., the minimum inhibitory concentration (MIC) for one or potentially all acceptable antimicrobial agents against a given pathogen.

Without wishing to limit the present invention to any theory or mechanism, a reason there is a range of probabilities (e.g., other than just 0 or 1) may be due to the fact that there are multiple variants of each pathogenic species in the overall population due to slight genetic modifications over time. A result may be that some species variants are inhibited at a given concentration while others are resistant at any viable concentration. Although the identity of the pathogenic species may be known, in some embodiments, the particular variants can be harder to characterize and identify and thus may not be known. Using the known population statistics on resistance, the number of wells that must be analyzed in the ABX plate can be minimized.

As a non-limiting example, the ABX plate may include 2-3 concentrations of each antimicrobial agent (or combination of antimicrobial agents) to be tested so as to allow for determination of the MIC. If the probability of resistance to a given antimicrobial agent at the highest concentration is high (but less than 100%, or 1) for a particular pathogen in a sample, then it may be most efficient to test this highest concentration first. If resistance is shown, which is the expected outcome, this resistance will eliminate that antimicrobial agent as a candidate without the need to test the other one or more smaller concentrations. If susceptible at that concentration, then the analysis can continue with the next lower concentration until the MIC is determined.

To identify the MIC for a pathogen-antimicrobial combination with a low p_{R} even at the lowest concentration, the course may be to test the lowest concentration well first. If susceptible, which is the expected outcome, then there is no reason to test the other one or more concentrations as the MIC has been established. If the p_{R} values of a pathogen across a range of concentrations of an antimicrobial agent are such that 0≤p_{R}≤1, then, in the case of 3 concentrations, it may be the best course to start with the middle concentration. Or, in the general case of n concentrations, the best course may be to start with the concentration that is most likely to sit at the edge of the resistance/susceptibility probability border and then follow with the increased or decreased concentration depending on the detection of resistance or susceptibility respectively in order to establish the MIC.

The present invention includes the possibility that the infection in the sample is a polymicrobial infection, e.g., two or more different infectious agents. In some embodiments, the p_{R}s are calculated based on the identity of the two or more different infectious agents and the susceptibility and resistance data of the combination of the infectious agents.

The present invention is not limited to the use of just the overall population statistics. Conditional probabilities can be used to further minimize the number of wells needed to be tested. For example, there may be some variations in pathogens that make them more resistant or susceptible to the mechanism of a certain antimicrobial action. For example, the p_{R} of a pathogen against a certain antimicrobial agent at its highest concentration may be, as an example, 0.5 (for this example, referred to as p_{R}(B)). If it is measured to be resistant or susceptible at that highest concentration, that data may help provide some information about what variant it is (even if it is not named), and it may thereafter be considered a variant that is now known to be resistant or susceptible based on the observed measure.

In the database (as described herein, e.g., for calculating p_{R} values), it may also be possible to calculate the conditional probability of resistance of the infectious agent(s) to a certain antimicrobial agent (A) at a certain concentration if the resistance to another antimicrobial agent (B) at a given concentration is known (for this example referred to as p_{R}(A|B)). In some embodiments, the conditional probability (probability of it being resistant to A given it is known that it is resistant to B) may be different than the overall observed probability p_{R}(A) due to factors that relate them (rather than being independent) such as, but not limited to, that the compounds in a class of antimicrobial agents may have similar inhibitory mechanisms and may be similarly affected by the change in the variant among other factors. For a given pathogen, identifying these antimicrobial agents (B) for which the conditional probabilities of other antimicrobial agents inhibiting that pathogen are changed dependent on the result can provide leverage to improve the efficiency of the plate analysis. For example, in some embodiments, instead of traversing the plate as indicated above, it may be best to target the antimicrobial agents that provide the most leverage in understanding the true probabilities of resistance against other agents given its susceptibility or resistance and then use that new information to optimize the analysis (minimize the wells visited/analyzed) as discussed above.

### Optional Automated System Components

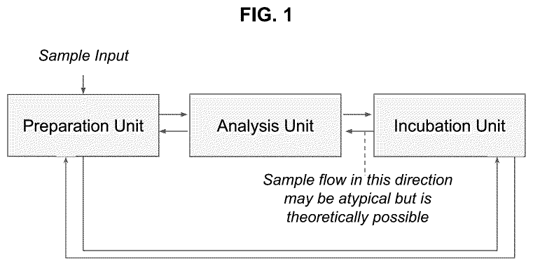

As shown in FIG. 1, the system may feature a preparation unit, e.g., a component of the system that prepares a sample for analysis (e.g., spin, wash, resuspend, etc.); an analysis unit, e.g., a flow cytometry system, a component of the system that may identify one or more infectious agents in the sample, quantify one or more infectious agents in the sample, identify one or more markers on or in the infectious agents in the sample, etc.; and/or an incubation unit, e.g., a component of the system that may function to store, hold, retain, or promote the growth of the sample (e.g., the infectious agents in the sample), such as an incubator. The preparation unit and/or analysis unit and/or incubation unit may be operatively linked, e.g., wherein a sample can be moved from one unit to another as is appropriate. In certain embodiments, the sample is preprocessed prior to insertion into the system.

As an example, a sample may be inserted into the preparation unit. From the preparation unit, the sample may move to the incubation unit. In certain embodiments, the sample moves from the incubation unit back to the preparation unit. The sample may then move from the preparation unit to the analysis unit to be analyzed. In some embodiments, the sample moves from the analysis unit back to the preparation unit. There may be certain embodiments wherein circumstances allow for the sample to move from the incubation unit to the analysis unit directly.

The systems of the present invention also feature components for transporting the sample or a portion thereof from one unit to another, e.g., from the preparation unit to the analysis unit, from the analysis unit to the incubation unit, from the preparation unit to the incubation unit, from the incubation unit to the analysis unit, etc.

The systems of the present invention may also provide reagents, e.g., buffers, growth media, dyes, labels, etc., for various steps, tests, and methods associated with the preparation unit, analysis unit (e.g., flow cytometry unit), and incubation unit.

The systems of the present invention also feature components for storing, manipulating, and administering said reagents, e.g., buffers, growth media, dyes, labels, etc.

The present invention is not limited to the particular fluorophores disclosed herein (or particular fluorophore conjugates), and alternative fluorophores are well known to one of ordinary skill in the art and within the scope of this invention.

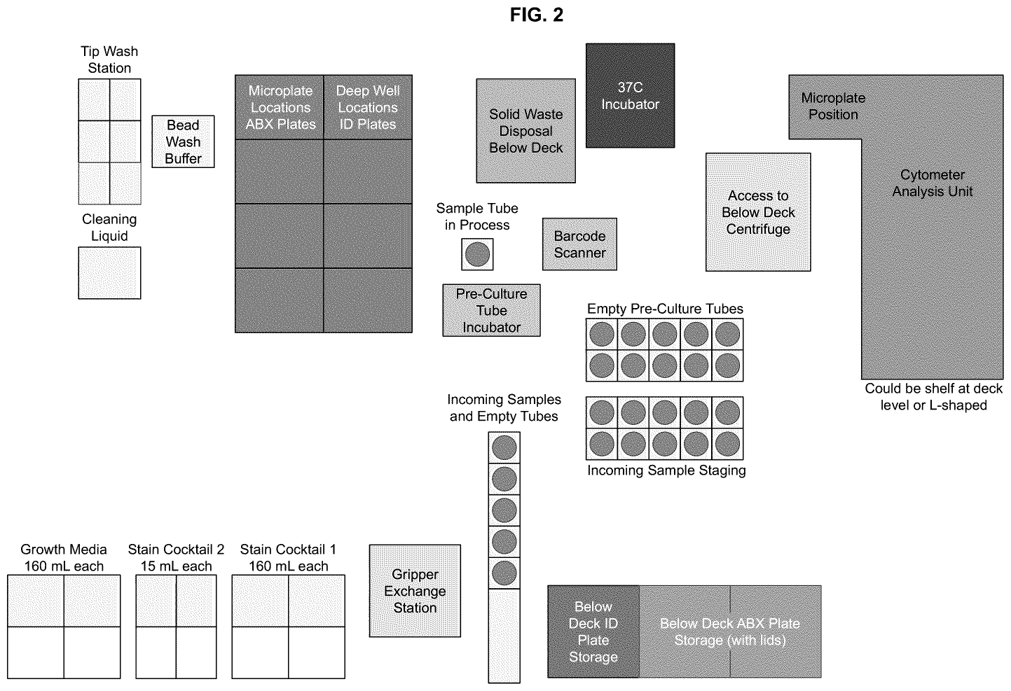

FIG. 2 shows a non-limiting example of a system of the present invention and a plurality of components and reagents included therein. For example, the system in FIG. 2 features growth media containers (e.g., various for different media), stain cocktail containers (e.g., various), cleaning liquid container(s), buffer containers (e.g., various, e.g., bead wash buffer, sample wash buffer, etc.), a tip wash station, a waste disposal tank, a bar code scanner, one or more incubators, stages or locations for samples or multiwell plates, a centrifuge, a cytometer analysis unit, storage (e.g., for ID plates, ABX plates, sample tubes, tips, etc.), etc.

The systems of the present invention also feature computer-implemented instructions for performing the automated methods herein. The systems of the present invention also feature computer-implemented instructions for analyzing signals obtained from one or more sensors and/or detectors associated with the systems.

Without wishing to limit the present invention to any theory or mechanism, it is believed that the systems and methods of the present invention may allow for the detection of at least 20 different target particles (e.g., cells or other particles such as but not limited to infectious agents), or at least 25 target particles, at least 30 target particles, at least 35 target particles, at least 40 target particles, etc. The system may feature the use of at least 20 different antibodies or binding agents, or at least 25, at least 30, at least 35, etc., each specific for a particular target particle in the sample. The system can also feature the detection of at least 40 different target particles (thus at least 40 different antibodies) using 5-30 labels or dyes.

The systems may be capable of at least 10,000 events per second, allowing for rapid and comprehensive analysis of samples. The present invention is not limited to 10,000 events per second. For example, in certain embodiments, the event rate may be higher than 10,000 events per second, e.g., 20,000 events per second, 30,000 events per second, more than 30,000 events per second, etc.

In some embodiments, the system can process at least 2 samples per day. In some embodiments, the system can process at least 5 samples per day. In some embodiments, the system can process at least 10 samples per day. In some embodiments, the system can process at least 15 samples per day. In some embodiments, the system can process at least 20 samples per day. In some embodiments, the system can process at least 25 samples per day. In some embodiments, the system can process at least 30 samples per day. In some embodiments, the system can process at least 35 samples per day. In some embodiments, the system can process at least 40 samples per day. In some embodiments, the system can process at least 45 samples per day. In some embodiments, the system can process at least 50 samples per day. In some embodiments, the system can process at least 55 samples per day. In some embodiments, the system can process at least 60 samples per day. In some embodiments, the system can process at least 65 samples per day. In some embodiments, the system can process at least 70 samples per day. In some embodiments, the system can process at least 75 samples per day. In some embodiments, the system can process at least 80 samples per day. In some embodiments, the system can process at least 85 samples per day. In some embodiments, the system can process at least 90 samples per day. In some embodiments, the system can process at least 95 samples per day. In some embodiments, the system can process at least 100 samples per day.

### Preparation Unit

The system of the present invention may comprise a preparation unit, e.g, a component of the system that prepares a sample for flow cytometry analysis in the analysis unit and/or incubation in the incubation unit. In certain embodiments, a raw sample is inserted directly into the system. In some embodiments, the sample is processed in some manner prior to insertion into the system.

For example, in some embodiments, samples are cleaned and concentrated. In certain embodiments, the samples are spun to create a pellet, washed, and resuspended in a medium prior to being transported to the analysis unit. Methods for cleaning and concentrating samples are known to one of ordinary skill in the art. In certain embodiments, the preparation unit comprises a centrifuge, a holding tank for wash buffer, and a holding tank for resuspension buffer. In some embodiments, the preparation unit comprises a magnetic bead separation rack. The preparation unit may further comprise a sample input component for accepting raw samples. The preparation unit further comprises tubing and/or hydraulics for aspirating media and/or wash buffer, and/or for introducing wash buffer and/or resuspension buffer to the pelleted samples. The preparation unit is not limited to the aforementioned components and/or configurations.

The preparation unit may allow for concentration of the sample and subsequent dilution. In some embodiments, the preparation unit provides the concentration of the sample. In some embodiments, the analysis unit provides the concentration of the sample. Knowledge of the initial concentration of the resuspended sample may help allow for the determination of antibiotic susceptibility and resistance of the sample (e.g., after processing in the incubation unit, for example).

### Analysis Unit

The system of the present invention comprises an analysis unit, e.g., a component of the system that identifies one or more infectious agents, and/or quantifies one or more infectious agents, etc. For example, the analysis unit may comprise one or more flow cytometers or flow cytometry-like devices. In some embodiments, the systems comprise a plurality of flow cytometry-like devices, e.g., two or more, three or more, four or more, etc. For example, the system may comprise three flow devices.

As previously discussed, flow cytometry can be used to measure one or more optical or electrical parameters of infectious agents that pass through a light beam (e.g., a laser). For example, a fluid sample to be analyzed is introduced from a sample tube into or near the center of a faster flowing stream of sheath fluid. The sample is compressed, causing the infectious agents in the sample volume to be columnated and be delivered to the center of the measuring point in an examination zone. A laser beam is focused on the infectious agents as they pass through the examination zone. Detectors that are optically connected to the examination zone interrogate signals from this zone on one or more detection channels. When an infectious agent in the flow stream is struck by the laser beam, certain signals are generated and sensed by detectors. The detectors utilize a plurality of detection channels and there may be multiple lasers of differing wavelengths spatially separated along the flow path to create multiple examination zones. Non-limiting examples of signals include forward scatter intensity, which provides information concerning the size of individual infectious agents, side scatter intensity, which provides information regarding the granularity (relative size, proportions, and refractive properties) of the infectious agents; and fluorescence emissions from one or more fluorescent dyes and/or fluorescent molecules associated with the infectious agents.

The system comprises components for measuring and/or introducing reagents to the sample. For example, the analysis unit may comprise a component for introducing one or a combination of antibodies (or binding fragments) conjugated with detectable labels or dyes, wherein the antibodies or binding fragments are specific for a single infectious agent. The conjugated antibodies (or binding fragments) allow for specific identification of the infectious agents.

In some embodiments, the system uses at least 5 conjugated antibodies (or binding fragments) for the identification of 5 infectious agents. In some embodiments, the system uses at least 10 conjugated antibodies (or binding fragments) for the identification of 10 infectious agents. In some embodiments, the system uses at least 20 conjugated antibodies (or binding fragments) for the identification of 20 infectious agents. In some embodiments, the system uses at least 30 conjugated antibodies (or binding fragments) for the identification of 30 infectious agents. In some embodiments, the system uses at least 40 conjugated antibodies (or binding fragments) for the identification of 40 infectious agents.

In certain embodiments, the systems of the present invention are configured to detect and identify small particles such as viruses. Methods and systems for viral detection using flow cytometry (e.g., flow virometry) are well known to one of ordinary skill in the art. In certain embodiments, the systems use labeled (e.g., fluorescently labeled) specific binding agents (e.g., antibodies, antibody fragments, etc.) for specific detection of viral particles. The present invention is not limited to the use of labeled specific binding agents and may include staining procedures or other methods for specific viral detection.

The present invention is not limited to the detection of specific infectious agents in the sample. In some embodiments, the systems of the present invention are configured to enumerate the infectious agents present in the sample. In some embodiments, the systems of the present invention are configured to distinguish (and quantitate) live and dead infectious agents present in the sample. Methods and reagents for enumerating infectious agents such as bacteria are well known as one of ordinary skill in the art. For example, commercially available reagents for enumerating bacteria include nucleic acid stains that penetrate both Gram-positive and Gram-negative bacteria, e.g., SYTO BC dye (ThermoFisher Scientific). Methods and reagents for distinguishing (and quantitating) live and dead infectious agents such as bacteria are well known to one of ordinary skill in the art. For example, commercially available reagents include propidium iodide, SYTO 9 dye (ThermoFisher Scientific), redox indicators for determining whether or not the cells are alive, e.g., CellROX (ThermoFisher Scientific), etc. The present invention is not limited to the aforementioned reagents.

In some embodiments, the systems are configured to identify active infections using markers for red blood cells (RBCs). In some embodiments, the systems are configured to detect other cell types and/or other particles. For example, in some embodiments, the systems are configured to detect white blood cells.

The system may be designed to determine which elements (e.g., which well of the microplate) to analyze based on ID, prior sample data (e.g., from a database), and/or the previous well's results. This helps minimize the time needed for analysis.

### Incubation Unit

The system of the present invention comprises an incubation unit, e.g., a component of the system that may function to store, hold, retain, or promote the growth of the sample (e.g., the infectious agents in the sample). For example, the incubation unit may comprise one or a combination of incubators to allow for growth of infectious agents in the sample. The incubators may be set to facilitate growth under particular conditions. In some embodiments, the incubation unit comprises an incubator in aerobic conditions. In some embodiments, the incubation unit comprises an incubator in anaerobic conditions.

Samples may be incubated in the incubation unit for a time frame. In some embodiments, the time frame is dependent on which infectious agents are detected by the analysis unit. In some embodiments, the time frame is 1 minute. In some embodiments, the time frame is 5 minutes. In some embodiments, the time frame is 8 minutes. In some embodiments, the time frame is 10 minutes. In some embodiments, the time frame is 20 minutes. In some embodiments, the time frame is 30 minutes. In some embodiments, the time frame is 60 minutes. In some embodiments, the time frame is 1.5 hours. In some embodiments, the time frame is 2 hours. In some embodiments, the time frame is 2.5 hours. In some embodiments, the time frame is 3 hours. In some embodiments, the time frame is 4 hours. In some embodiments, the time frame is 5 hours. In some embodiments, the time frame is 6 hours. In some embodiments, the time frame is 6 or more hours. In some embodiments, the time frame is 9 or more hours. In some embodiments, the time frame is 12 hours. In some embodiments, the time frame is 12 or more hours. In some embodiments, the time frame is from 1 to 10 minutes. In some embodiments, the time frame is from 5 to 20 minutes. In some embodiments, the time frame is from 10 to 30 minutes. In some embodiments, the time frame is from 10 to 60 minutes. In some embodiments, the time frame is from 30 to 60 minutes. In some embodiments, the time frame is from 1 to 2 hours. In some embodiments, the time frame is from 2 to 4 hours. In some embodiments, the time frame is from 4 to 6 hours. In some embodiments, the time frame is from 6 to 9 hours. In some embodiments, the time frame is from 9 to 12 hours. In some embodiments, the time frame is more than 12 hours.

As an example, a portion of the sample (e.g., a portion with a predetermined number of organisms) may be inoculated in a growth medium and incubated in aerobic conditions at a particular temperature, e.g., 37 degrees, for a time frame. In some embodiments, the incubated sample may be incubated in anaerobic conditions at a particular temperature, e.g., 37 degrees, for a time frame.

In certain embodiments, a portion of the sample may be inoculated in a growth medium comprising an antibiotic or antimicrobial agent. In certain embodiments, a portion of the sample may be inoculated in a growth medium comprising one or more antibiotics or antimicrobial agents. In certain embodiments, a portion of the sample may be inoculated in a growth medium comprising two or more antibiotics or antimicrobial agents. In certain embodiments, a portion of the sample may be inoculated in a growth medium comprising three or more antibiotics or antimicrobial agents.

In some embodiments, the preparation unit comprises components that measure and/or aliquot portions of the sample into one or more wells of a multiwell plate; alternatively, in some embodiments, the incubation unit comprises components that measure and/or aliquot portions of the sample into one or more wells of a multiwell plate. The incubation unit may further comprise components for moving the multiwell plates in and out of the appropriate incubator, or receiving the sample from the preparation unit, or transporting the sample to the preparation unit, etc.

The systems of the present invention are configured to determine the antibiotic susceptibility of infectious agents present in the sample.

Samples, after incubation, may be redirected to the analysis unit for testing. In certain embodiments, the samples return to the preparation unit prior to being redirected to the analysis unit.

In some embodiments, the sample comprises a single infectious agent. In some embodiments, the sample comprises 2 or more infectious agents. In some embodiments, the sample comprises 3 or more infectious agents. In some embodiments, the sample comprises 4 or more infectious agents. In some embodiments, the sample comprises 5 or more infectious agents. In some embodiments, the sample comprises 6 or more infectious agents. In some embodiments, the sample comprises 7 or more infectious agents. In some embodiments, the sample comprises 8 or more infectious agents. In some embodiments, the sample comprises 9 or more infectious agents. In some embodiments, the sample comprises 10 or more infectious agents.

In some embodiments, the system can process a sample (referring to the identification process, e.g., obtain the sample, subject the sample to the preparation unit and analysis unit, and provide information related to the identification and quantification of infectious agents present in the sample, if any) within 15 minutes. In some embodiments, the system can process a sample (e.g., obtain the sample, subject the sample to the preparation unit and analysis unit, and provide information related to the identification and quantification of infectious agents present in the sample, if any) within 20 minutes. In some embodiments, the system can process a sample (e.g., obtain the sample, subject the sample to the preparation unit and analysis unit, and provide information related to the identification and quantification of infectious agents present in the sample, if any) within 30 minutes. In some embodiments, the system can process a sample (e.g., obtain the sample, subject the sample to the preparation unit and analysis unit, and provide information related to the identification and quantification of infectious agents present in the sample, if any) within 45 minutes. In some embodiments, the system can process a sample (e.g., obtain the sample, subject the sample to the preparation unit and analysis unit, and provide information related to the identification and quantification of infectious agents present in the sample, if any) within one hour. In some embodiments, the system can process a sample (e.g., obtain the sample, subject the sample to the preparation unit and analysis unit, and provide information related to the identification and quantification of infectious agents present in the sample, if any) within two hours. In some embodiments, the system can process a sample (e.g., obtain the sample, subject the sample to the preparation unit and analysis unit, and provide information related to the identification and quantification of infectious agents present in the sample, if any) within three hours. In some embodiments, the system can process a sample (e.g., obtain the sample, subject the sample to the preparation unit and analysis unit, and provide information related to the identification and quantification of infectious agents present in the sample, if any) within four hours. In some embodiments, the system can process a sample (e.g., obtain the sample, subject the sample to the preparation unit and analysis unit, and provide information related to the identification and quantification of infectious agents present in the sample, if any) within five hours. In some embodiments, the system can process a sample (e.g., obtain the sample, subject the sample to the preparation unit and analysis unit, and provide information related to the identification and quantification of infectious agents present in the sample, if any) within six hours. In some embodiments, the system can process a sample (e.g., obtain the sample, subject the sample to the preparation unit and analysis unit, and provide information related to the identification and quantification of infectious agents present in the sample, if any) within seven hours. In some embodiments, the system can process a sample (e.g., obtain the sample, subject the sample to the preparation unit and analysis unit, and provide information related to the identification and quantification of infectious agents present in the sample, if any) within eight hours. In some embodiments, the system can process a sample (e.g., obtain the sample, subject the sample to the preparation unit and analysis unit, and provide information related to the identification and quantification of infectious agents present in the sample, if any) within nine hours. In some embodiments, the system can process a sample (e.g., obtain the sample, subject the sample to the preparation unit and analysis unit, and provide information related to the identification and quantification of infectious agents present in the sample, if any) within ten hours. In some embodiments, the system can process a sample (e.g., obtain the sample, subject the sample to the preparation unit and analysis unit, and provide information related to the identification and quantification of infectious agents present in the sample, if any) within twelve hours. In some embodiments, the system can process a sample (e.g., obtain the sample, subject the sample to the preparation unit and analysis unit, and provide information related to the identification and quantification of infectious agents present in the sample, if any) within 24 hours.

In some embodiments, the system can process a sample, e.g., obtain the sample, subject the sample to the preparation unit and analysis unit, and provide information related to the identification and quantification of infectious agents present in the sample and if positive generate the AST results within 4-6 hours.

In some embodiments, the system can process at least 1 sample per day. In some embodiments, the system can process at least 2 samples per day. In some embodiments, the system can process at least 3 samples per day. In some embodiments, the system can process at least 4 samples per day. In some embodiments, the system can process at least 5 samples per day. In some embodiments, the system can process at least 8 samples per day. In some embodiments, the system can process at least 10 samples per day. In some embodiments, the system can process at least 15 samples per day. In some embodiments, the system can process at least 20 samples per day. In some embodiments, the system can process at least 25 samples per day. In some embodiments, the system can process at least 50 samples per day.

### EXAMPLE 1

The following is a non-limiting example of the present invention. It is to be understood that said example is not intended to limit the present invention in any way. Equivalents or substitutes are within the scope of the present invention.

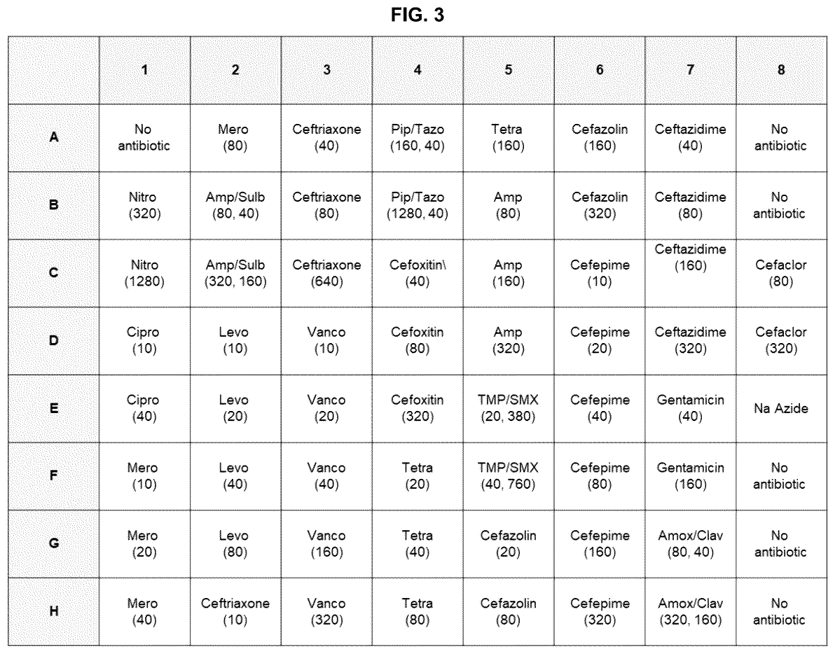

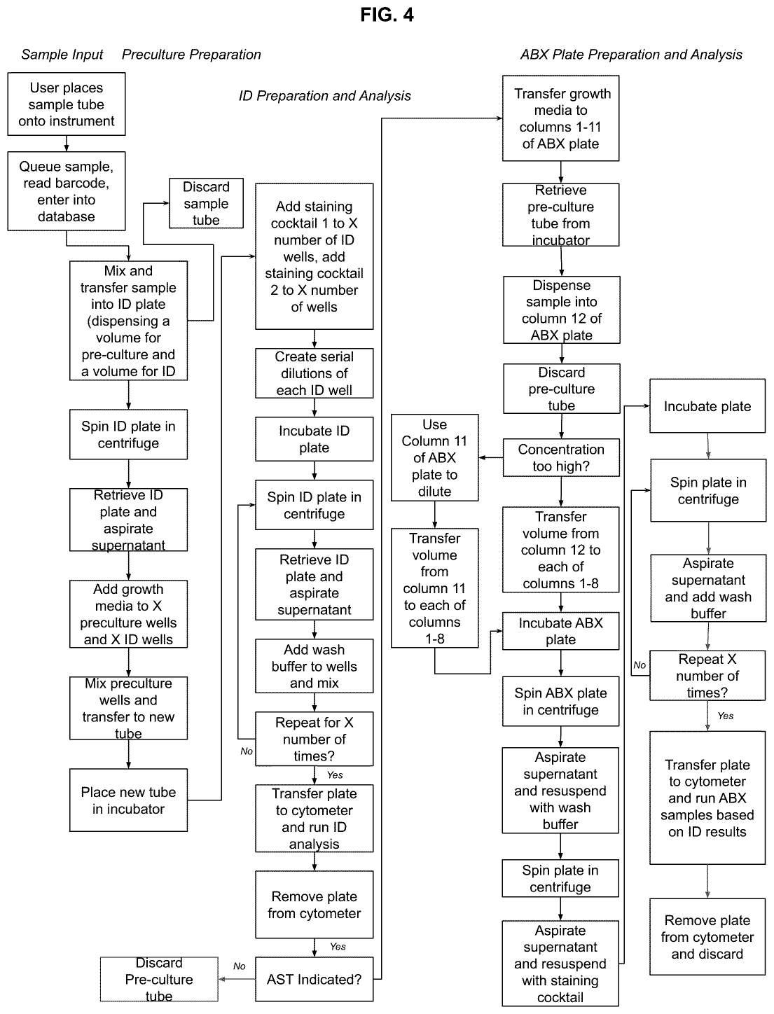

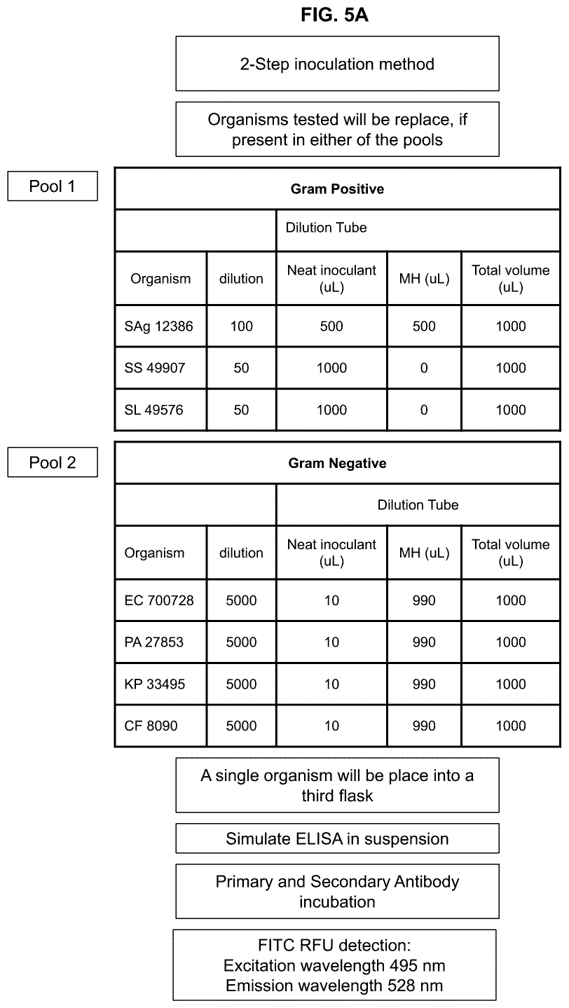

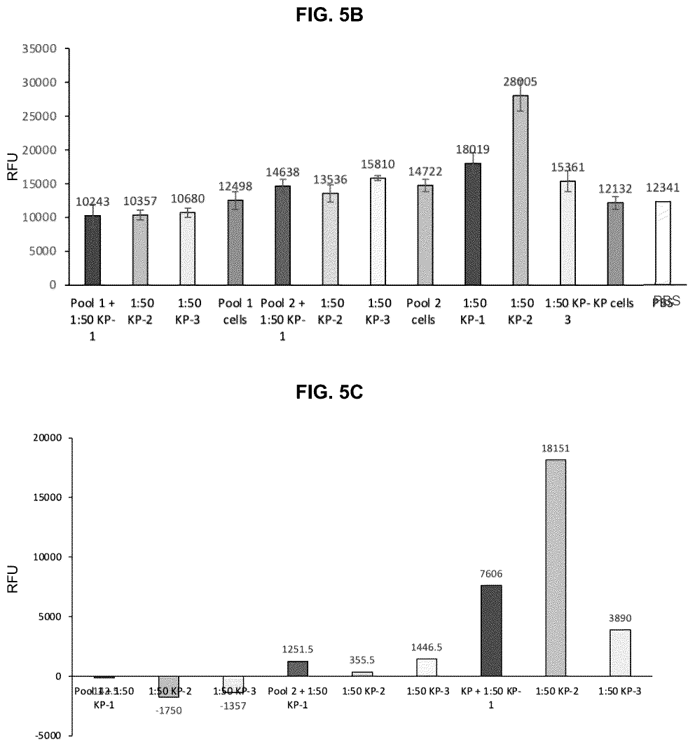

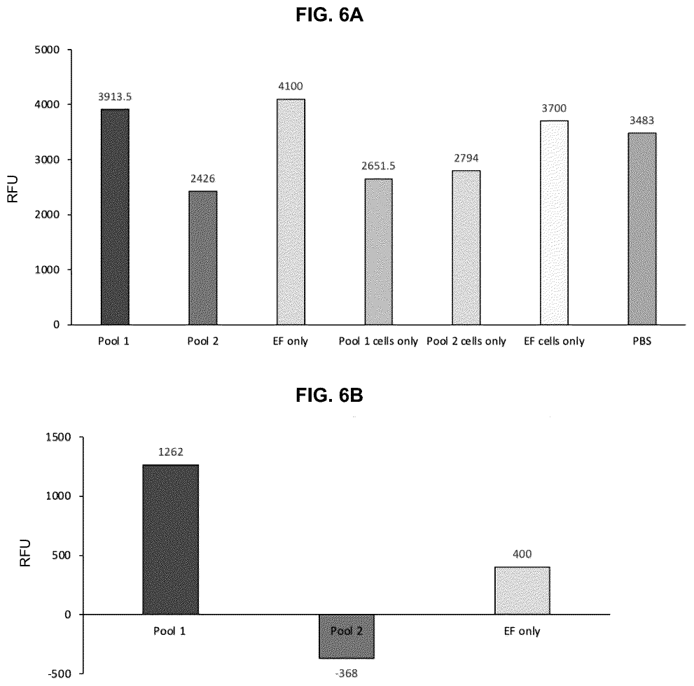

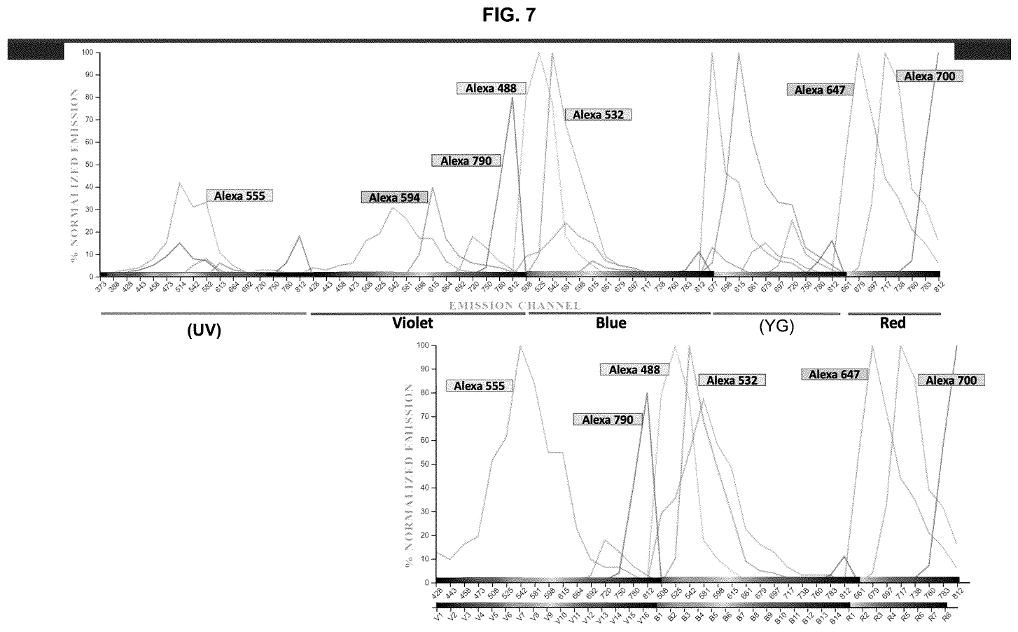

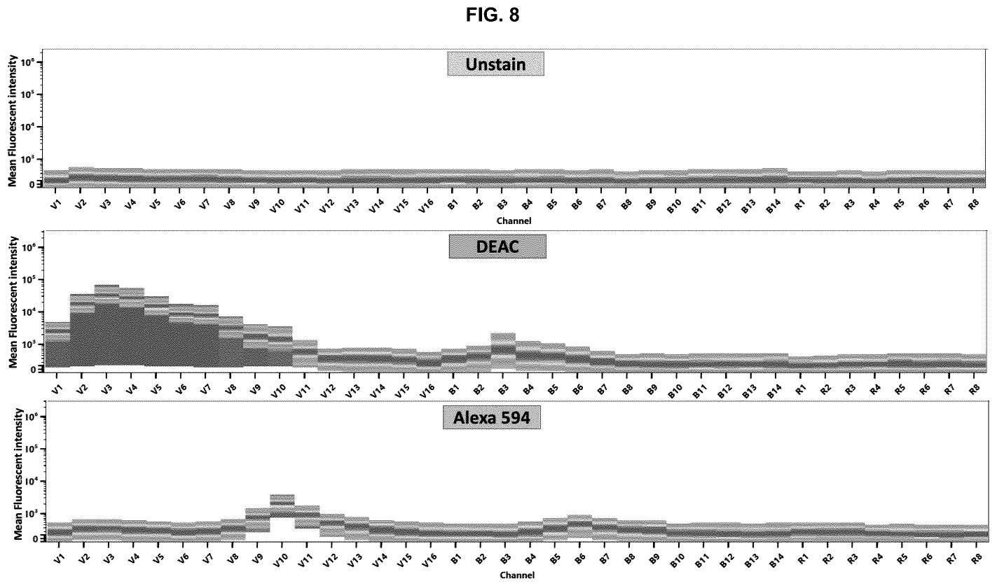

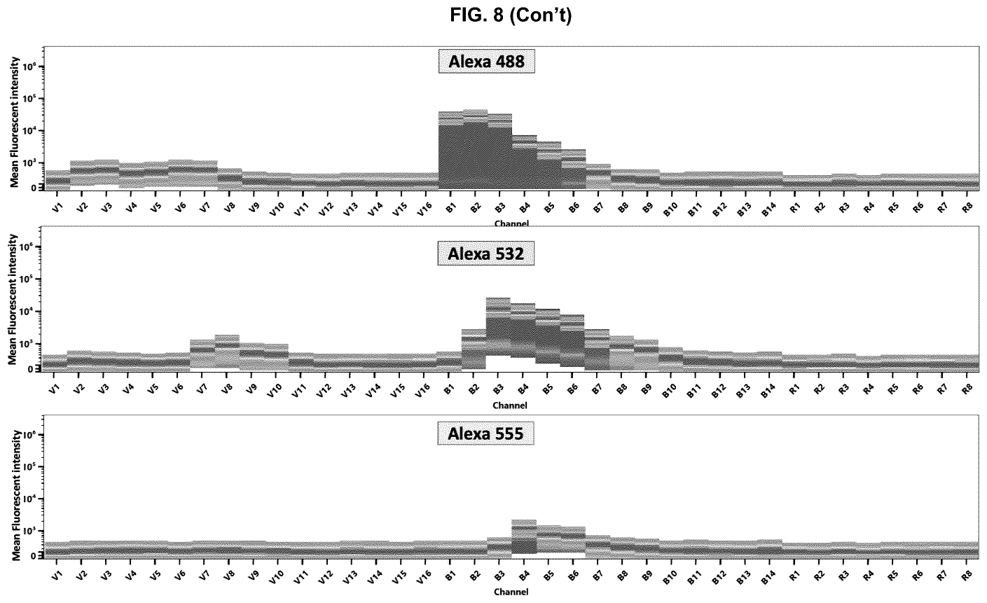

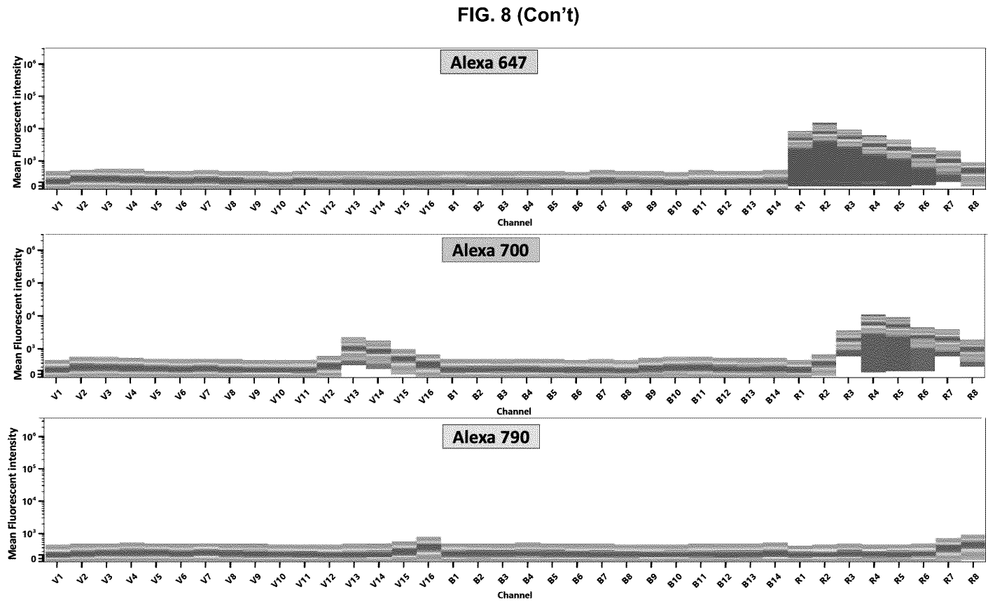

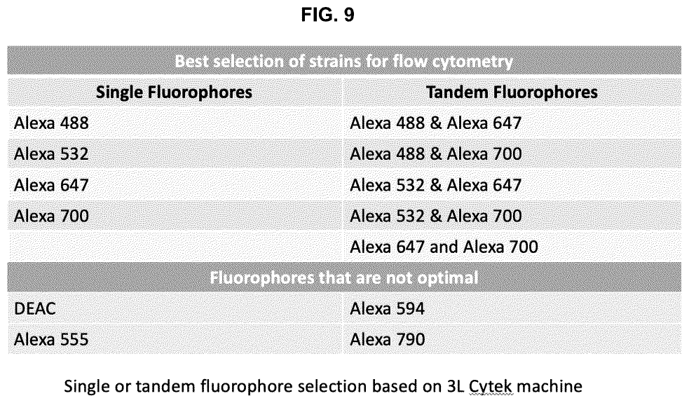

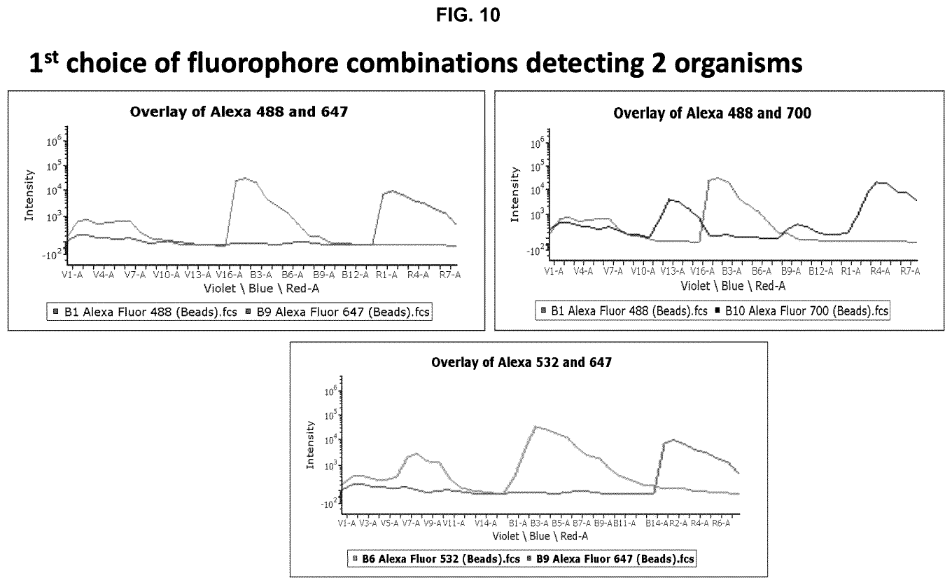

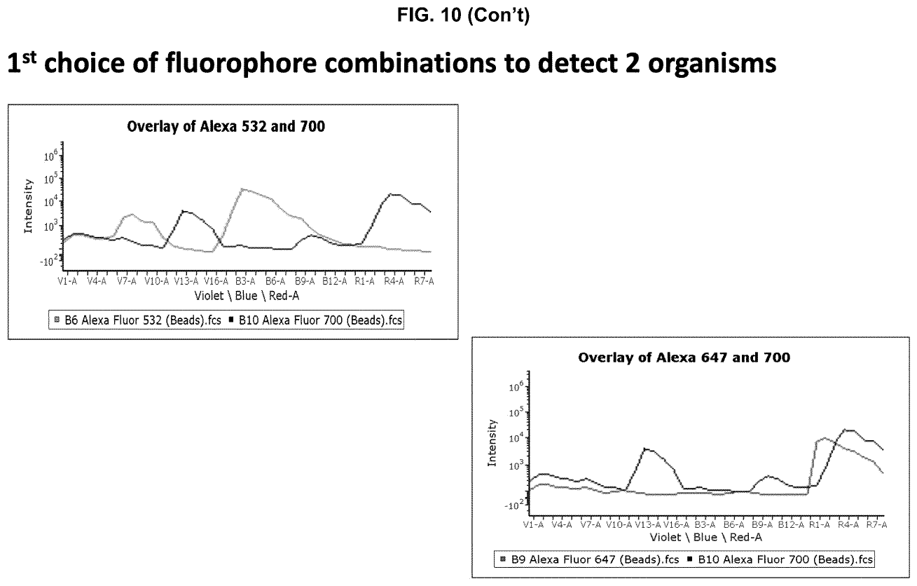

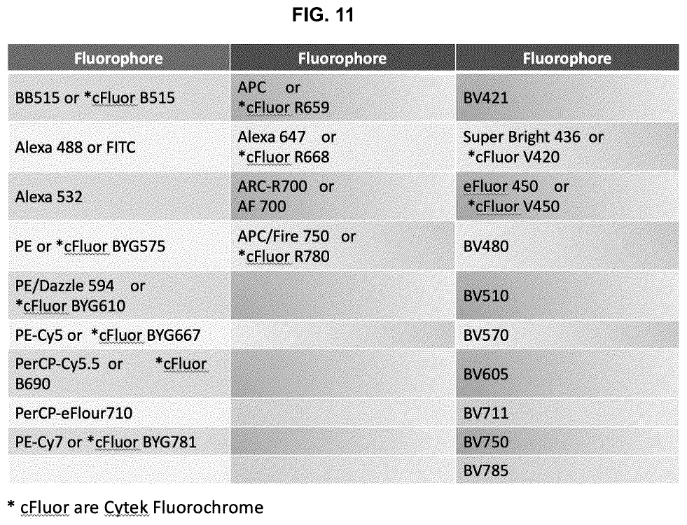

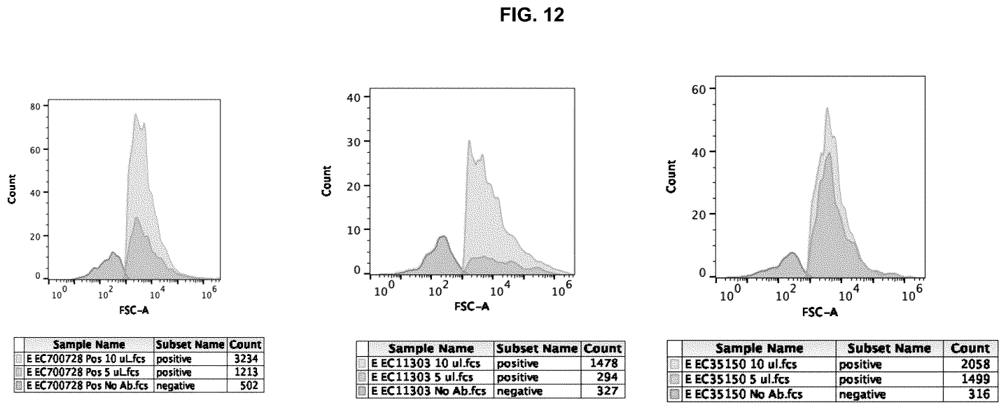

FIG. 3 and FIG. 4 show and describe embodiments of the system of the present invention. For example, a sample, e.g., contained in a tube or vessel, is placed onto the instrument, e.g., a stage. In certain embodiments, the sample tubes feature a barcode for identification. The system may feature a barcode reader that reads the barcode of the sample and enters it into the database (e.g., of the computer system, e.g., the processing and memory units, etc.).

After the sample has been identified in the computer system, the sample may be moved from the stage to a different location where it is mixed and aliquoted to wells of a multiwell plate. The present invention is not limited to a particular type or size of multiwell plate. In certain embodiments, the system houses the multiwell plates in a storage container and obtains one of the plates as needed. In certain embodiments, a user loads the system with the multiwell plate to be used for the specific sample when needed. The sample tube (and any leftover sample) may be discarded after the appropriate amount of sample has been aliquoted into the wells of the multiwell plate.

Next, the plate may be transported from its location to a centrifuge to be spun, after which the plate is removed from the centrifuge and transported to a location where supernatant is aspirated (and discarded) and growth media is added to the appropriate wells (e.g., some of the wells) of the plate in preparation for preculture incubation.

In certain embodiments, one or a combination of wells resuspended with growth media are mixed and then transferred to a new sample tube (optionally, certain wells may be combined together and transferred into one new sample tube), e.g., a pre-culture tube. In certain embodiments, the pre-culture tube is transported to the incubator for a period of time.

Next, the system may aliquot staining cocktails to the appropriate wells of the multiwell plate and perform serial dilutions for each set of cocktails used to provide more dilute samples in the case that the infectious agents are initially present in too high of a concentration for analysis. In certain embodiments, the plate is incubated at room temperature for a period of time. When ready, the plate may be centrifuged, the supernatant aspirated, and wash solution added; this set of steps may be repeated. Next, the plate may be transferred to the flow cytometer to determine the identities of the infectious agents in the sample. For example, the system may start with analysis of the most dilute wells for each cocktail used and either use that well for the analysis or use said well for determining which well is best for analysis. The results of the flow cytometry analysis may help determine if the sample should be subjected to antibiotic susceptibility testing.

If antibiotic susceptibility testing (AST) is not needed, the sample pre-culture tube (in the incubator) may be discarded.

If antibiotic susceptibility testing (AST) is needed, growth media may be plated into various wells of a multiwell plate (e.g., an ABX plate). The pre-culture tube from the incubator is accessed and the sample is aliquoted into various wells as appropriate. If the concentration of the sample is too high, the sample may be diluted prior to inoculation of the wells. The ABX plate may be returned to the incubator for a period of time.

After incubation is complete, the ABX plate is retrieved and subjected to centrifugation, a wash step, and resuspension; this series of steps may be repeated if needed. Staining cocktails may be added to particular wells as appropriate and mixed. The plate may be incubated at room temperature for a period of time. In certain embodiments, the ABX plate is retrieved and subjected to centrifugation, a wash step, and resuspension; this series of steps may be repeated. After said preparation, the ABX plate is transferred to the cytometer for analysis for susceptibility.

Note that the system may be configured to minimize the number of wells analyzed by the flow cytometer based on the initial identification of the infectious agent and prioritization of wells analyzed by leveraging results from all wells analyzed for that sample. The end result is a determination of MIC for the various antimicrobial agents or combinations thereof that inhibit the growth of the infectious agents in the sample.

### EXAMPLE 2

The following is a non-limiting example of the present invention. It is to be understood that said example is not intended to limit the present invention in any way. Equivalents or substitutes are within the scope of the present invention.

An example of a system described herein is configured to detect a panel of organisms. For example, the organisms capable of being detected are one or a combination of: Acinetobacter baumannii, Actinobaculum schaalii, Aerococcus urinae, Alloscardovia omnicolens, Candida albicans, Candida glabrata, Candida parapsilosis, Citrobacter koseri, Citrobacter freundii, Corynebacterium riegelii, Enterococcus faecalis, Enterobacter aerogenes, Escherichia coli, Klebsiella oxytoca, Klebsiella pneumoniae, Morganella morganii, Mycoplasma hominis, Pantoea agglomerans, Proteus mirabilis, Providencia stuartii, Pseudomonas aeruginosa, Serratia marcescens, Staphylococcus aureus, Staphylococcus epidermidis, Staphylococcus haemolyticus, Staphylococcus lugdunensis, Staphylococcus saprophyticus, Streptococcus agalactiae, Streptococcus anginosus, Streptococcus oralis, Streptococcus pasteuranus, and Ureaplasma urealyticum.

For the purpose of this example only, the sample is a urine sample being tested for a urinary tract infection.

For the purpose of this example only, the following antibody conjugates are used: monoclonal antibody against E. coli antigen conjugated with Super Bright 436; monoclonal antibody against S. oralis antigen conjugated with Alexa Fluor 488; monoclonal antibody against C. freundii antigen conjugated with PE-Cyanine7; monoclonal antibody against E. faecalis antigen conjugated with Super Bright 600; monoclonal antibody against S. aureus antigen conjugated with NovaFluor Yellow 610; monoclonal antibody against C. alibicans antigen conjugated with Alexa Fluor 647; monoclonal antibody against A. schaalii antigen conjugated with Alexa Fluor 700; monoclonal antibody against S. epidermidis antigen conjugated with SuperBright 702; monoclonal antibody against K. pneumoniae antigen conjugated with APC-eFluor 780.

As previously discussed, the present invention is not limited to the aforementioned fluorophores or aforementioned antibody-fluorophore conjugates, and alternative fluorophores are well known to be an ordinary skill in the art and are within the scope of this invention.

As previously discussed, the present invention is not limited to analysis of bacteria. Other infectious agents or other targets of interest (not limited to infectious agents), including antigens, particles, viruses, other cells, etc., may also be subject to analysis using the system of the present invention.

After the sample has been identified in the computer system, the sample is moved from the stage to a different location where it is mixed and aliquoted to four of the wells of a multiwell deep well plate.

Next, the plate is transported from its location to a centrifuge to be spun, after which the plate is removed from the centrifuge and transported to a location where supernatant is aspirated (and discarded) and growth media is added to two of the wells of the plate in preparation for pre-culture incubation.

The wells resuspended with growth media are mixed and then transferred to a single new sample tube, e.g., a pre-culture tube. The pre-culture tube is transported to the incubator for a period of time.

Next, the system aliquots two staining cocktails separately into each of the two remaining sample wells and two each of four additional empty wells of the multiwell plate and performs serial dilutions of the two sample wells for each set of cocktails used to provide more dilute samples in the case that the infectious agents are initially present in too high of a concentration for analysis. The plate is incubated at room temperature for a period of time. After incubation, the plate is centrifuged, the supernatant aspirated, and wash solution added; this set of steps is repeated two more times.

Next, the plate is transferred to the flow cytometer for determining identities of the infectious agents in the sample. The system starts with analysis of the most dilute wells for each cocktail used and either use that well for the analysis or use said well for determining which well is best for analysis and then analyze that well. The result of this flow cytometry analysis indicates the following infectious agents are detected at infectious levels, e.g., at concentrations above the infection threshold: E. coli at 8×10⁴ cells/ml and E. faecalis at 6×10⁶ cells/ml.

Previously collected data in a database is used to predict the probability of susceptibility against the various concentrations and types of antimicrobial agents in the ABX plate to determine which wells are optimal to test and which are likely unnecessary to analyze.

The system will next perform antibiotic susceptibility testing (AST). The system features a multiwell plate (ABX plate) wherein a plurality of the wells house various individual antimicrobial agents at various particular concentrations or a combination of antimicrobial agents at particular concentrations. For example, the ABX plate may feature a series of concentrations of one or more of the following antimicrobial agents: Ciprofloxacin, Levofloxacin, Tetracycline, Cefaclor, Nitrofurantoin, Ampicillin, Sulfamethoxazole/Trimethoprim, Ceftriaxone, Gentamicin, Cefazolin, Cefepime, Cefoxitin, Ceftazidime, Meropenem, and/or Vancomycin. The ABX plate may feature a series of concentrations of one or more of the following combinations of antimicrobial agents: Piperacillin/Tazobactam, Amoxicillin/Clavulanate, and/or Ampicillin/Sulbactam. A non-limiting example of an ABX plate with its series of concentrations of particular antimicrobial agents is shown in FIG. 3.

Growth media is plated into various wells of a multiwell plate (e.g., an ABX plate). The pre-culture tube from the incubator is accessed and the sample is allotted into various wells as appropriate. If the concentration of the sample is too high, the sample may be diluted prior to inoculation of the wells. The ABX plate may be returned to the incubator for a period of time.

After incubation is complete, the ABX plate is retrieved and subjected to centrifugation, a wash step, and resuspension. A staining cocktail is added to particular wells as appropriate and mixed. The plate is incubated at room temperature for a period of time. The ABX plate is retrieved and subjected to centrifugation, a wash step, and resuspension; this series of steps is repeated twice. After said preparation, the ABX plate is transferred to the cytometer for analysis for susceptibility.

Next, the system analyzes the results of the AST. The AST analysis provides information related to the antimicrobial agents to which the sample is susceptible and the antimicrobial agents to which the sample is resistant. The AST analysis also provides minimum inhibitory concentrations (MICs).

### EXAMPLE 3

The following is a non-limiting example of the present invention. It is to be understood that said example is not intended to limit the present invention in any way. Equivalents or substitutes are within the scope of the present invention.

The following example provides additional non-limiting examples of antibody conjugates: monoclonal antibody against Streptococcus anginosus antigen conjugated with StarBright Violet 515; monoclonal antibody against Viridans Group Streptococcus antigen conjugated with FITC; monoclonal antibody against M. genitalium antigen conjugated with YFP; monoclonal antibody against P. stuartii antigen conjugated with PE-Alexa Fluor 750; monoclonal antibody against A. baumannii antigen conjugated with DyLight 650; monoclonal antibody against C. glabrata antigen conjugated with mCherry; monoclonal antibody against HHV-7 antigen conjugated with PerCP-Cy5.5; monoclonal antibody against Adenovirus antigen conjugated with StarBright Blue 700; monoclonal antibody against K. oxytoca antigen conjugated with Brilliant Violet 711.

Additional non-limiting examples of antibody conjugates may include: monoclonal antibody against Chlamydia antigen conjugated with PE-Cyanine 5.5; monoclonal antibody against S. marcescens antigen conjugated with EGFP; monoclonal antibody against BK Virus antigen conjugated with StarBright Violet 440; monoclonal antibody against C. glabrata antigen conjugated with Pacific Blue; monoclonal antibody against Coagulase-negative Staphylococcusantigen conjugated with PerCP; monoclonal antibody against M. hominis antigen conjugated with Alexa Fluor 405; monoclonal antibody against T. vaginalis antigen conjugated with APC-Cy7; monoclonal antibody against U. urealyticum antigen conjugated with RFP; monoclonal antibody against P. mirabilia antigen conjugated with DyLight 550.

### EXAMPLE 4

The following is a non-limiting example of the present invention. It is to be understood that said example is not intended to limit the present invention in any way. Equivalents or substitutes are within the scope of the present invention.

An example protocol in the application of the systems herein includes, but is not limited to: (1) introducing a sample to the system for identification by placing the sample contained within a tube (e.g., initial sample tube) onto the system and scanning the identifying barcode and entering it into a system database; (2) performing pre-culture preparation and incubation of the sample by mixing and transferring the sample contained within the initial sample tube to an identification plate, centrifuging the identification plate and disposing of the supernatant, adding fresh growth media to each well and mixing and dispensing the contents of the pre-culture wells into a clean tube (e.g., pre-culture tube) for incubation at 37 degrees Celsius for a period of time; (3) performing sample identification preparation steps by staining the sample wells and executing a serial dilution before allowing the identification plate to incubate at room temperature for a period of time followed by washing and mixing the sample wells, repeating the sample preparation step 2-3 times in total; (4) performing sample analysis by transferring the identification plate to a flow cytometer and processing the first sample well (e.g., the most dilute sample) and depending on the result, performing analysis of additional sample wells to select the sample well with highest acceptable concentration to identify pathogens and their concentration and whether additional ABX plate preparation is required; (5) if required, performing ABX plate preparation steps by inoculating a new ABX plate and placing it in an incubator at 37 degrees Celsius for a period of time before performing washing and staining steps; and (6) performing ABX plate analysis for antibiotic susceptibility by introducing the ABX plate to a flow cytometer and visiting ABX plate wells based on pathogen identification and antibiotic concentration. The present invention is not limited to said protocol disclosed herein.

### EMBODIMENTS

The following embodiments are intended to be illustrative only and not to be limiting in any way.

Embodiment 1: A system configured to analyze optical data captured by a flow cytometer, wherein the data captured by the flow cytometer is from a detection system therein that detects optical emission as a target particle passes through the flow cytometer, the optical emission being primarily derived from one or a combination of: (i) one or more fluorophores each conjugated to an antibody, the antibody being one of n antibody species each specific for one of n target particles, the one or more fluorophores being from a group of m total fluorophores used for labeling the n antibody species forming a pool of l antibody-fluorophore conjugates, and (ii) potential autofluorescence associated with the target particle, the system comprising: (a) a processor and (b) a memory component communicatively connected (e.g.,operatively connected; e.g., via a wire connection or wireless connection) to the processor and configured to store an application (e.g., computer readable instructions), the application (e.g., computer readable instructions) allows the processor to identify a dominant antibody-fluorophore conjugate on each target particle using an unmixing algorithm, the dominant antibody-fluorophore conjugate being an antibody-fluorophore conjugate that appears in abundance preferentially on the target particle due to its binding to the target particle; wherein the unmixing algorithm either: uses a single minimum abundance ratio (X) value using a smallest minimum value of X for any of the antibody-fluorophore conjugates constraining the solution to require a dominant antibody fluorophore-conjugate with abundance value of at least X times that of a next most abundant antibody-fluorophore conjugate as a requirement for an acceptable solution for a closed form unmixing algorithm or as a constraint on a numerical mixing algorithm; or partitions the analysis into n separate instances, wherein n is the number of possible target particles that can be identified in the analysis, wherein each instance has a unique minimum abundance ratio requirement based on a predetermined value of X for each antibody-fluorophore conjugate, the minimum abundance ratio requirement being a requirement that a particular dominant antibody-fluorophore conjugate have an abundance value greater than X times that of a next most abundant antibody-fluorophore conjugate in order to be a correct solution for a closed form expression and wherein the minimum abundance ratio requirement is an additional constraint for a numerical solution with target particle identification based on the instance with the smallest residual error.

Embodiment 2: The system of embodiment 1, wherein the abundance value for the dominant antibody-fluorophore conjugate is required to be above a predetermined threshold level of fluorescence.

Embodiment 3: The system of embodiment 1 or 2, wherein the residual error of the numerical analysis must be below a threshold value or below a specific fraction of a next closest residual error value in order to be considered a valid unambiguous result.

Embodiment 4: The system of any of embodiments 1-3, wherein the system is capable of detecting and quantifying a plurality of different targets within a single sample.

Embodiment 5: The system of embodiment 4, wherein the plurality of targets is at least 10 different targets.

Embodiment 6: The system of embodiment 4, wherein the plurality of targets is at least 20 different targets.

Embodiment 7: The system of any of embodiments 1-6, wherein the minimum abundance ratio (X) is at least >1 for each antibody-fluorophore conjugate.

Embodiment 8: The system of any of embodiments 1-6, wherein the minimum abundance ratio (X) is from 1 to 10 for at least one antibody-fluorophore conjugate.

Embodiment 9: The system of any of embodiments 1-6, wherein the minimum abundance ratio (X) is from 3 to 10 for at least one antibody-fluorophore conjugate.

Embodiment 10: The system of any of embodiments 1-6, wherein the minimum abundance ratio (X) is from 5 to 20 for at least one antibody-fluorophore conjugate.

Embodiment 11: The system of any of embodiments 1-10, wherein the solutions are constrained so that all abundance values are greater than zero.

Embodiment 12: The system of any of embodiments 1-11, wherein each target particle has a predetermined autofluorescence signature.

Embodiment 13: The system of embodiment 12, wherein each autofluorescence signature is added as an additional fluorophore paired with the respective minimum abundance ratio (X) for the target particle in that instance.

Embodiment 14: The system of embodiment 12 or 13, wherein each autofluorescence signature is added as an additional fluorophore for purposes of fitting measured spectra in the numerical analysis.

Embodiment 15: The system of any of embodiments 1-14, wherein each antibody is conjugated with a single unique fluorophore.

Embodiment 16: The system of any of embodiments 1-14, wherein the pool of l antibody-fluorophore conjugates comprises n different antibodies each labeled with a unique fluorophore.

Embodiment 17: The system of any of embodiments 1-14, wherein the pool of l antibody-fluorophore conjugates comprises n different antibodies, a portion of which are each labeled with one fluorophore and a portion of which are each labeled with two or more fluorophores.

Embodiment 18: The system of embodiment 17, wherein in the pool of l antibody-fluorophore conjugates at least one antibody has at least 2 different antibody conjugates each of which are labeled with one or a combination from a set of r different fluorophores, and said fluorophores are also used as labels on other antibodies for different target particles.

Embodiment 19: The system of embodiment 17 or 18, wherein the number of particles that the system can identify using a subset of r fluorophores is the sum of each of the terms r!/((r−k)!*k!) for all k from 1 to r, wherein r can be less than or equal to m.

Embodiment 20: The system of any of embodiments 17-19, wherein the system can identify 3 distinct target particles using 2 fluorophores.

Embodiment 21: The system of any of embodiments 17-19, wherein the system can identify 7 distinct target particles using 3 fluorophores.

Embodiment 22: The system of any of embodiments 19-21, wherein the minimum abundance ratio requirement is approximately X/k for each of k fluorophores used for the particular antibody and wherein X is the minimum abundance ratio when all of the particular antibody is labeled with a single fluorophore.

Embodiment 23: The system of any of embodiments 1-22, wherein the flow cytometer is a spectral flow cytometry system.

Embodiment 24: The system of any of embodiments 1-23, wherein the system is communicatively connected (e.g.,operatively connected; e.g., via a wire connection or wireless connection) to the flow cytometer.

Embodiment 25: The system of any of embodiments 1-24, wherein the system is integrated with the flow cytometer.

Embodiment 26: The system of any of embodiments 1-25, wherein the target particle is a cell or particle.

Embodiment 27: The system of embodiment 26, wherein the cell or particle is or is associated with an infectious agent.

Embodiment 28: The system of embodiment 27, wherein the infectious agent is a bacterium, a virus, a fungus, or a parasite.

Embodiment 29: The system of embodiment 27 or 28, wherein the antibodies are each specific for an infectious agent.

Embodiment 30: The system of any of embodiments 1-29, wherein the antibodies are monoclonal antibodies.

Embodiment 31: A system configured to analyze optical data captured by a flow cytometer, wherein the data captured by the flow cytometer is from a detection system therein that detects optical emission as a target particle passes through the flow cytometer, the optical emission being primarily derived from one or a combination of: (i) one or more fluorophores each conjugated to an antibody, the antibody being one of n antibody species each specific for one of n target particles, the one or more fluorophores being from a group of m total fluorophores used for labeling the n antibody species forming a pool of l antibody-fluorophore conjugates, and (ii) potential autofluorescence associated with the target particle, the system comprising: (a) a processor and (b) a memory component communicatively connected (e.g., operatively connected; e.g., via a wire connection or wireless connection) to the processor and configured to store an application (e.g., computer readable instructions), the application (e.g., computer readable instructions) allows the processor to identify a dominant antibody-fluorophore conjugate on each target particle using an unmixing algorithm, the dominant antibody-fluorophore conjugate being an antibody-fluorophore conjugate that appears in abundance preferentially on the target particle due to its binding to the target particle; wherein the unmixing algorithm partitions the analysis into n separate instances, wherein n is the number of possible target particles that can be identified in the analysis, wherein each instance has a unique minimum abundance ratio requirement based on a predetermined value of X for each antibody-fluorophore conjugate, the minimum abundance ratio requirement being a requirement that a particular dominant antibody-fluorophore conjugate have an abundance value greater than X times that of a next most abundant antibody-fluorophore conjugate in order to be a correct solution for a closed form expression and wherein the minimum abundance ratio requirement is an additional constraint for a numerical solution with target particle identification based on the instance with the smallest residual error.

Embodiment 32: A system configured to analyze optical data captured by a flow cytometer, wherein the data captured by the flow cytometer is from a detection system therein that detects optical emission as a target particle passes through the flow cytometer, the optical emission being primarily derived from one or a combination of: ((i) one or more fluorophores each conjugated to an antibody, the antibody being one of n antibody species each specific for one of n target particles, the one or more fluorophores being from a group of m total fluorophores used for labeling the n antibody species forming a pool of l antibody-fluorophore conjugates, and (ii) potential autofluorescence associated with the target particle, the system comprising: (a) a processor; and (b) a memory component communicatively connected (e.g., operatively connected; e.g., via a wire connection or wireless connection) to the processor and configured to store an application (e.g., computer readable instructions), the application (e.g., computer readable instructions) allows the processor to identify a dominant antibody-fluorophore conjugate on each target particle using an unmixing algorithm, the dominant antibody-fluorophore conjugate being an antibody-fluorophore conjugate that appears in abundance preferentially on the target particle due to its binding to the target particle; wherein the unmixing algorithm uses a single minimum abundance ratio (X) value using a smallest minimum value of X for any of the antibody-fluorophore conjugates constraining the solution to require a dominant antibody fluorophore-conjugate with abundance value of at least X times that of a next most abundant antibody-fluorophore conjugate as a requirement for an acceptable solution for a closed form unmixing algorithm or as a constraint on a numerical mixing algorithm.

Embodiment 33: An antibody-fluorophore conjugate for use in identifying one or more target particles or target cells in a sample with a system according to any of embodiments 1-32, wherein the antibody of the antibody-fluorophore conjugate is specific for a particular target particle or target cell and the fluorophore of the antibody-fluorophore conjugate is a fluorophore with an emission spectra further distinguished from other similar fluorophore spectra in the analysis by the autofluorescence of the target particle or target cell.

Embodiment 34: An antibody-fluorophore conjugate for use in identifying one or more target particles or target cells in a sample with a system according to any of embodiments 1-32, wherein the antibody of the antibody-fluorophore conjugate is specific for a particular target particle or target cell and the fluorophore of the antibody-fluorophore conjugate is selected so that it is: (a) a weak or dim fluorophore if it typically binds to its target particle or target cell at a relatively high binding density; (b) a strong or bright fluorophore if it typically binds to its target particle or target cell at a relatively low binding density; (c) a medium intensity fluorophore if the typical binding to the target particle or target cell is not at a relatively high binding density and not at a relieve low binding density.

Embodiment 35: An antibody-fluorophore conjugate for use in identifying one or more infectious agents in a sample with the system according to any of embodiments 1-32, wherein the antibody of the antibody-fluorophore conjugate is specific for a particular infectious agent and the fluorophore of the antibody-fluorophore conjugate is selected so that it is: (a) a weak or dim fluorophore if it typically binds to its infectious agent at a relatively high binding density; (b) a strong or bright fluorophore if it typically binds to its infectious agent at a relatively low binding density; (c) a medium intensity fluorophore if the typical binding to the infectious agent is not at a relatively high binding density and not at a relieve low binding density.

Embodiment 36: A flow cytometry panel for use with the system according to any of embodiments 1-32, wherein the panel comprises a plurality of antibody-fluorophore conjugates according to one or a combination of embodiments 33-35.

Embodiment 37: An automated system for identifying one or more infectious agents in a sample, said system comprising: (a) a flow cytometry unit; and (b) a processing system for analyzing data from the flow cytometry unit according to any of embodiments 1-32.

Embodiment 38: The system of embodiment 37 further comprising an antibiotic susceptibility testing (AST) unit for determining susceptibility and resistance of the one or more infectious agents in the sample to one or more concentrations of one or more antimicrobial agents.

Embodiment 39: The system of embodiment 38, wherein the AST unit comprises a multiwell plate wherein a plurality of wells are loaded with a particular concentration of one or more antimicrobial agents.

Embodiment 40: The system of embodiment 39, wherein the AST unit further comprises a dispensing system for dispensing a portion of the sample into at least one well of the multiwell plate.

Embodiment 41: The system of any of embodiments 38-40, wherein the AST unit is communicatively connected (e.g.,operatively connected; e.g., via a wire connection or wireless connection) to the processing system, the processing system further comprises an application (e.g., computer readable instructions) stored by the memory for execution by the processor, the application (e.g., computer readable instructions) determines the order in which wells are analyzed for susceptibility or resistance.

Embodiment 42: The system of any of embodiments 30-41, wherein the application determines an order in which wells are analyzed that will likely result in the fewest wells needed to be analyzed in order to get all necessary information for providing a minimum inhibitory concentration (MIC) for one or more of the antimicrobial agents.

Embodiment 43: The system of any of embodiments 30-42, wherein the application (e.g., computer readable instructions) accesses a database comprising data that provides the application (e.g., computer readable instructions) a probability of resistance (p_{R}) for one or more concentrations of the one or more antimicrobial agents.

Embodiment 44: The system of embodiment 43, wherein the system determines which wells to analyze first based on the provided p_{R} values.

Embodiment 45: The system of any of embodiments 30-41, wherein the application (e.g., computer readable instructions) uses conditional probabilities to minimize the number of wells that need to be analyzed, wherein the application (e.g., computer readable instructions) calculates the probability of resistance of the one or more infectious agents to a certain antimicrobial agent (A) at a certain concentration based on the resistance of the one or more infectious agents to another antimicrobial agent (B) at a particular concentration.

Embodiment 46: The system of any of embodiments 37-45 further comprising an incubation unit comprising one or more incubators for housing and incubating a portion of the sample.

Embodiment 47: The system of embodiment 46 further comprising a preparation unit for accepting the sample and preparing the sample for processing through the flow cytometry unit.

Embodiment 48: The system of any of embodiments 37-47, wherein the system is for identifying an active infection with one or more infectious agents in the sample.

Embodiment 49: The system of embodiments 48, wherein the infectious agent is a bacteria, a virus, a fungus, or a parasite.

Embodiment 50: The system of embodiment 49, wherein the infectious agent is Acinetobacter baumannii, Actinotignum schaalii, Aerococcus urinae, Aerococcus urinae, Alloscardovia omnicolens, Candida albicans, Candida glabrata, Candida parapsilosis, Candida tropicalis, Chlamydia, Citrobacter freundii, Citrobacter koseri, Clostridium difficile, Corynebacterium riegelii, Klebsiella aerogenes, Enterococcus faecalis, Escherichia coli, Klebsiella oxytoca, Klebsiella pneumoniae, Morganella morganii, Mycobacterium tuberculosis, Mycoplasma genitalium, Mycoplasma hominis, Neisseria gonorrhoeae, Pantoea agglomerans, Proteus mirabilis, Providencia stuartii, Pseudomonas aeruginosa, Serratia marcescens, Staphylococcus aureus, coagulase-negative Staphylococcus, Streptococcus agalactiae, Streptococcus pyogenes, Viridans Group Streptococcus, Trichomonas vaginalis, Ureaplasma urealyticum, HHV-6, HHV-7, BK Virus, JC Virus, HSV 1&2, Adenovirus, or CMV.

Embodiment 51: The system of any of embodiments 37-50, wherein the system is configured for parallel processing of samples.

Embodiment 52: The system of any of embodiments 37-51, wherein the system is fully automated.

Embodiment 53: The system of any of embodiments 37-52, wherein the system processes the sample by dispensing portions into one or more multiwell plates.

Embodiment 54: The system of embodiment 53, wherein the system can transport the multiwell plate between the sample preparation unit and the incubator and the sample preparation unit and the analysis unit.

Embodiment 55: The system of any of embodiments 53-54, wherein the multiwell plate comprises wells having different concentrations of various antimicrobial agents to facilitate susceptibility analysis.

EMbodiment 56: The system of any of embodiments 37-55, wherein the system first identifies the infectious agent in the sample and optionally subsequently subjects the sample to antibiotic susceptibility testing.

Embodiment 57: The system of any of embodiments 37-56, wherein the system is configured to minimize the number of wells analyzed by the flow cytometer based on identification of the infectious agent and prioritization of wells analyzed.

Embodiment 58: The system of any of embodiments 37-57, wherein the sample is a urine sample, a blood sample, a CSF sample, a respiratory swap sample, a nasal swab sample, an abscess sample, an ascites sample, a cyst sample, a Lavine swab sample, a sepsis sample.

Embodiment 59: The system of any of embodiments 37-58, wherein the system is organized into a sample preparation unit for accepting the sample, an incubation unit, and an analysis unit, the analysis unit comprising the flow cytometry unit, wherein the sample can move between both the incubation unit and the sample processing unit, and the sample can move between the analysis unit and the sample processing unit.

Embodiment 60: The system of any of embodiments 37-59, wherein the system comprises one or more computer processing units.

Embodiment 61: The system of embodiment 60, wherein one or more of the computer processing units are operatively connected (e.g., communicatively connected; e.g., via a wire connection or wireless connection) to the sample processing unit, the incubation unit, the analysis unit, or a combination thereof.

Embodiment 62: The system of embodiment 60 or 61, wherein the computer processing unit comprises a microprocessor and one or more memory components, wherein at least one of the memory components comprises computer readable instructions for the microprocessor.

Embodiment 63: The system of embodiment 62, wherein the computer readable instructions allow the microprocessor to regulate movement of the sample within or between the sample processing unit, the incubation unit, and analysis unit.

Embodiment 64: The system of any of embodiments 60-63, wherein the computer processing unit is configured to analyze results from the flow cytometer so as to provide identity of one or more infectious agents in the sample.

Embodiments of the present invention can be freely combined with each other if they are not mutually exclusive.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

What is claimed is:

1. A system configured to analyze optical data captured by a flow cytometer, wherein the data captured by the flow cytometer is from a detection system therein that detects optical emission as a target particle passes through the flow cytometer, the optical emission being primarily derived from one or a combination of: (i) one or more fluorophores each conjugated to an antibody, the antibody being one of n antibody species each specific for one of n target particles, the one or more fluorophores being from a group of m total fluorophores used for labeling the n antibody species forming a pool of l antibody-fluorophore conjugates, and (ii) potential autofluorescence associated with the target particle, the system comprising:
a) a processor; and
b) a memory component communicatively connected to the processor and configured to store an application, the application allows the processor to identify a dominant antibody-fluorophore conjugate on each target particle using an unmixing algorithm, the dominant antibody-fluorophore conjugate being an antibody-fluorophore conjugate that appears in abundance preferentially on the target particle due to its binding to the target particle;
wherein the unmixing algorithm partitions the analysis into n separate instances, wherein n is the number of possible target particles that can be identified in the analysis, wherein each instance has a unique minimum abundance ratio requirement based on a predetermined value of X for each antibody-fluorophore conjugate, the minimum abundance ratio requirement being a requirement that a particular dominant antibody-fluorophore conjugate have an abundance value greater than X times that of a next most abundant antibody-fluorophore conjugate in order to be a correct solution for a closed form expression and wherein the minimum abundance ratio requirement is an additional constraint for a numerical solution with target particle identification based on the instance with the smallest residual error.

2. A system configured to analyze optical data captured by a flow cytometer, wherein the data captured by the flow cytometer is from a detection system therein that detects optical emission as a target particle passes through the flow cytometer, the optical emission being primarily derived from one or a combination of: ((i) one or more fluorophores each conjugated to an antibody, the antibody being one of n antibody species each specific for one of n target particles, the one or more fluorophores being from a group of m total fluorophores used for labeling the n antibody species forming a pool of l antibody-fluorophore conjugates, and (ii) potential autofluorescence associated with the target particle, the system comprising:
a) a processor; and
b) a memory component operatively connected to the processor and configured to store an application, the application allows the processor to identify a dominant antibody-fluorophore conjugate on each target particle using an unmixing algorithm, the dominant antibody-fluorophore conjugate being an antibody-fluorophore conjugate that appears in abundance preferentially on the target particle due to its binding to the target particle;
wherein the unmixing algorithm uses a single minimum abundance ratio (X) value using a smallest minimum value of X for any of the antibody-fluorophore conjugates constraining the solution to require a dominant antibody fluorophore-conjugate with abundance value of at least X times that of a next most abundant antibody-fluorophore conjugate as a requirement for an acceptable solution for a closed form unmixing algorithm or as a constraint on a numerical mixing algorithm.

3. A system configured to analyze optical data captured by a flow cytometer, wherein the data captured by the flow cytometer is from a detection system therein that detects optical emission as a target particle passes through the flow cytometer, the optical emission being primarily derived from one or a combination of: (i) one or more fluorophores each conjugated to an antibody, the antibody being one of n antibody species each specific for one of n target particles, the one or more fluorophores being from a group of m total fluorophores used for labeling the n antibody species forming a pool of l antibody-fluorophore conjugates, and (ii) potential autofluorescence associated with the target particle, the system comprising:
a) a processor; and
b) a memory component communicatively connected to the processor and configured to store an application, the application allows the processor to identify a dominant antibody-fluorophore conjugate on each target particle using an unmixing algorithm, the dominant antibody-fluorophore conjugate being an antibody-fluorophore conjugate that appears in abundance preferentially on the target particle due to its binding to the target particle;
wherein the unmixing algorithm either:
uses a single minimum abundance ratio (X) value using a smallest minimum value of X for any of the antibody-fluorophore conjugates constraining the solution to require a dominant antibody fluorophore-conjugate with abundance value of at least X times that of a next most abundant antibody-fluorophore conjugate as a requirement for an acceptable solution for a closed form unmixing algorithm or as a constraint on a numerical mixing algorithm; or
partitions the analysis into n separate instances, wherein n is the number of possible target particles that can be identified in the analysis, wherein each instance has a unique minimum abundance ratio requirement based on a predetermined value of X for each antibody-fluorophore conjugate, the minimum abundance ratio requirement being a requirement that a particular dominant antibody-fluorophore conjugate have an abundance value greater than X times that of a next most abundant antibody-fluorophore conjugate in order to be a correct solution for a closed form expression and wherein the minimum abundance ratio requirement is an additional constraint for a numerical solution with target particle identification based on the instance with the smallest residual error.

4. The system of claim 3, wherein the target particle is a cell or particle; wherein the cell or particle is or is associated with an infectious agent.

5. The system of claim 3, wherein the sample is a urine sample, a blood sample, a CSF sample, a respiratory swap sample, a nasal swab sample, an abscess sample, an ascites sample, a cyst sample, a Lavine swab sample, a sepsis sample.

6. The system of claim 3, wherein the system is organized into a sample preparation unit for accepting the sample, an incubation unit, and an analysis unit, the analysis unit comprising the flow cytometry unit, wherein a sample can move between both the incubation unit and the sample processing unit, and the sample can move between the analysis unit and the sample processing unit.

7. The system of claim 6, wherein the system comprises one or more computer processing units, wherein one or more of the computer processing units are communicatively connected to the sample processing unit, the incubation unit, the analysis unit, or a combination thereof, wherein the computer processing unit comprises a microprocessor and one or more memory components, wherein at least one of the memory components comprises computer readable instructions for the microprocessor, wherein the computer-readable instructions allow the microprocessor to regulate movement of the sample within or between the sample processing unit, the incubation unit, and analysis unit.

8. The system of claim 7, wherein the computer processing unit is configured to analyze results from the flow cytometer so as to provide identity of one or more infectious agents in the sample.

9. The system of claim 6, wherein the system processes the sample by dispensing portions of the sample into one or more multiwell plates, wherein the system can transport the one or more multiwell plates between the sample preparation unit and the incubator and the sample preparation unit and the analysis unit.

10. The system of claim 9, wherein the one or more multiwell plates comprise wells having different concentrations of various antimicrobial agents to facilitate susceptibility analysis.

11. The system of claim 3, wherein the system first identifies an infectious agent in the sample and optionally subsequently subjects the sample to antibiotic susceptibility testing.

12. A flow cytometry panel for use with the system according to claim 3, wherein the panel comprises a plurality of antibody-fluorophore conjugates according to one or a combination of:
a) an antibody-fluorophore conjugate for use in identifying one or more target particles or target cells in a sample, wherein the antibody of the antibody-fluorophore conjugate is specific for a particular target particle or target cell and the fluorophore of the antibody-fluorophore conjugate is a fluorophore with an emission spectra further distinguished from other similar fluorophore spectra in the analysis by the autofluorescence of the target particle or target cell; or
b) an antibody-fluorophore conjugate for use in identifying one or more target particles or target cells in the sample, wherein the antibody of the antibody-fluorophore conjugate is specific for a particular target particle or target cell and the fluorophore of the antibody-fluorophore conjugate is selected so that it is: (a) a weak or dim fluorophore if it typically binds to its target particle or target cell at a relative high binding density; (b) a strong or bright fluorophore if it typically binds to its target particle or target cell at a relative low binding density; (c) a medium intensity fluorophore if the typical binding to the target particle or target cell is not at a relative high binding density and not at a relieve low binding density.

13. An automated system for identifying one or more infectious agents in a sample, said system comprising:
a) a flow cytometry unit; and
b) a processing system for analyzing data from the flow cytometry unit according to claim 3.

14. The system of claim 3 further comprising an antibiotic susceptibility testing (AST) unit for determining susceptibility and resistance of one or more infectious agents in the sample to one or more concentrations of one or more antimicrobial agents, wherein the AST unit comprises a multiwell plate wherein a plurality of wells are loaded with a particular concentration of one or more antimicrobial agents.

15. The system of claim 14, wherein the AST unit further comprises a dispensing system for dispensing a portion of the sample into at least one well of the multiwell plate.

16. The system of claim 14, wherein the AST unit is communicatively connected to the processing system, the processing system further comprises an application stored by the memory for execution by the processor, the application determines the order in which wells are analyzed for susceptibility or resistance.

17. The system of claim 3, wherein the application determines an order in which wells are analyzed that will likely result in the fewest wells needed to be analyzed in order to get all necessary information for providing a minimum inhibitory concentration (MIC) for one or more of the antimicrobial agents.

18. The system of claim 3, wherein the application uses conditional probabilities to minimize the number of wells that need to be analyzed, wherein the application calculates the probability of resistance of the one or more infectious agents to a certain antimicrobial agent (A) at a certain concentration based on the resistance of the one or more infectious agents to another antimicrobial agent (B) at a particular concentration.

19. The system of claim 3, wherein the system is configured for parallel processing of samples.

20. The system of claim 3, wherein the system is fully automated.
